(19) United States
(12) Patent Application Publication
(10) Pub. No.: US 2020/0060523 A1
(43) Pub. Date: Feb. 27, 2020

(54) MEDICAL SUPPORT ARM SYSTEM AND CONTROL DEVICE

(71) Applicant: Sony Corporation, Tokyo (JP)

(72) Inventors: Yasuhiro MATSUDA, Tokyo (JP); Atsushi MIYAMOTO, Kanagawa (JP); Kenichiro NAGASAKA, Tokyo (JP); Masaru USUI, Tokyo (JP); Yohei KURODA, Tokyo (JP); Daisuke NAGAO, Kanagawa (JP); Jun ARAI, Tokyo (JP); Tetsuharu FUKUSHIMA, Tokyo (JP)

(73) Assignee: Sony Corporation, Tokyo (JP)

(21) Appl. No.: 16/487,436

(22) PCT Filed: Feb. 19, 2018
(86) PCT No.: PCT/JP2018/005610

§ 371 Date: Aug. 21, 2019

(30) Foreign Application Priority Data
Feb. 28, 2017 (JP) 2017-036260

(51) Int. Cl. A61B 1/00; B25J 13/06; B25J 9/16; A61B 34/30; A61B 34/20; A61B 1/313

(52) U.S. Cl. CPC A61B 1/00149; B25J 13/06; B25J 9/1689; A61B 1/00059; A61B 1/00045; A61B 90/25; A61B 1/00177; A61B 34/30; A61B 34/20; A61B 1/3132; A61B 1/00179

(57) ABSTRACT

Provision of a technology for controlling an arm to maintain hand-eye coordination is desirable in a case of using the arm for supporting an oblique-viewing endoscope.

Provided is a medical support arm system including an articulated arm configured to support a scope that acquires an image of an observation target in an operation field, and a control unit configured to control the articulated arm on the basis of a relationship between a real link corresponding to a lens barrel axis of the scope and a virtual link corresponding to an optical axis of the scope.

### TECHNICAL FIELD

The present disclosure relates to a medical support arm system and a control device.

### BACKGROUND ART

Conventionally, for example, Patent Document 1 describes, in a medical observation device, a configuration including an imaging unit that captures an image of an operation site, and a holding unit to which the imaging unit is connected and provided with rotation axes in an operable manner with at least six degrees of freedom, in which at least two axes, of the rotation axes, are active axes controlled to be driven on the basis of states of the rotation axes, and at least one axis, of the rotation axes, is a passive axis rotated according to a direct operation with contact from an outside.

### CITATION LIST

### Patent Document

### Patent Document 1: International Publication No. 2016/017532

### SUMMARY OF THE INVENTION

### Problems to be Solved by the Invention

By the way, in an endoscope inserted into a human body, even if there is an obstacle in front of an observation target, the observation target can be observed without being blocked by the obstacle by using an oblique-viewing endoscope. However, maintaining hand-eye coordination is required in a case of using the oblique-viewing endoscope.

Therefore, provision of a technology for controlling an arm to maintain hand-eye coordination is desirable in a case of using the arm for supporting the oblique-viewing endoscope.

### Solutions to Problems

According to the present disclosure, provided is a medical support arm system including an articulated arm configured to support a scope that acquires an image of an observation target in an operation field, and a control unit configured to control the articulated arm on the basis of a relationship between a real link corresponding to a lens barrel axis of the scope and a virtual link corresponding to an optical axis of the scope.

### Effects of the Invention

As described above, according to the present disclosure, in a case of using an arm for supporting an oblique-viewing endoscope, the arm can be controlled to maintain hand-eye coordination.

Note that the above-described effect is not necessarily limited, and any of effects described in the present specification or other effects that can be grasped from the present specification may be exerted in addition to or in place of the above-described effect.

### BRIEF DESCRIPTION OF DRAWINGS

FIG. 1 is a diagram illustrating an example of a schematic configuration of an endoscopic surgical system to which the technology according to the present disclosure is applicable.

FIG. 2 is a block diagram illustrating an example of functional configurations of a camera head and a CCU illustrated in FIG. 1.

FIG. 3 is a perspective view illustrating a configuration example of a medical support arm device according to an embodiment of the present disclosure.

FIG. 4 is an explanatory diagram for describing ideal joint control according to an embodiment of the present disclosure.

FIG. 5 is a functional block diagram illustrating a configuration example of a robot arm control system according to an embodiment of the present disclosure.

FIG. 6 is a schematic view illustrating a configuration of an oblique-viewing endoscope according to an embodiment of the present disclosure.

FIG. 7 is a schematic view illustrating an oblique-viewing endoscope and a forward-viewing endoscope in comparison.

FIG. 8 is a schematic diagram illustrating a state in which an oblique-viewing endoscope is inserted through an abdominal wall into a human body, and an observation target is observed.

FIG. 9 is a schematic diagram illustrating a state in which an oblique-viewing endoscope is inserted through an abdominal wall into a human body, and an observation target is observed.

FIG. 10 is a view for describing an optical axis of an oblique-viewing endoscope.

FIG. 11 is a view for describing an operation of the oblique-viewing endoscope.

FIG. 12 is a diagram for describing modeling and control.

FIG. 13 is a diagram illustrating an example of link configurations in a case where extension of whole body coordination control is applied to a six-axis arm and an oblique-viewing endoscope unit.

FIG. 14 is a diagram illustrating an example of link configurations in a case where extension of whole body coordination control is applied to a six-axis arm and an oblique-viewing endoscope unit.

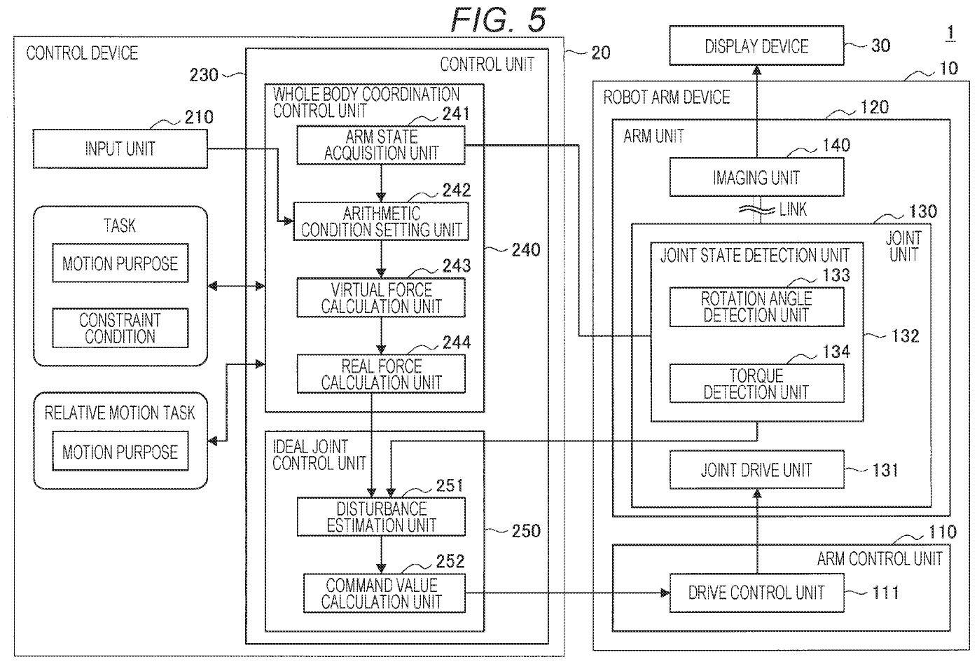

FIG. 5A is a diagram illustrating a first example of an oblique-viewing endoscope applicable to the present embodiment.

FIG. 15B is a diagram illustrating the first example of an oblique-viewing endoscope applicable to the present embodiment.

FIG. 16A is a diagram illustrating a second example of an oblique-viewing endoscope applicable to the present embodiment.

FIG. 16B is a diagram illustrating the second example of an oblique-viewing endoscope applicable to the present embodiment.

FIG. 17A is a diagram illustrating a third example of an oblique-viewing endoscope applicable to the present embodiment.

FIG. 17B is a diagram illustrating the third example of an oblique-viewing endoscope applicable to the present embodiment.

FIG. 18 is a diagram for describing an oblique angle fixed oblique-viewing endoscope.

FIG. 19 is a diagram for describing update of a virtual rotary link in consideration of a zoom operation of the oblique angle fixed oblique-viewing endoscope.

FIG. 20 is a diagram for describing update of a virtual rotary link in consideration of a zoom operation of an oblique angle variable oblique-viewing endoscope.

### MODE FOR CARRYING OUT THE INVENTION

Favorable embodiments of the present disclosure will be described in detail with reference to the appended drawings. Note that, in the present specification and drawings, redundant description of configuration elements having substantially the same functional configuration is omitted by providing the same sign.

Note that the description will be given in the following order.

1. Configuration Example of Endoscopic System

2. Specific Configuration Example of Support Arm Device

3. Basic Configuration of Oblique-viewing endoscope

4. Control of Arm Supporting Oblique-viewing endoscope According to Present Embodiment

5. Setting of Virtual Link

6. Conclusion

### 1. CONFIGURATION EXAMPLE OF ENDOSCOPIC SYSTEM

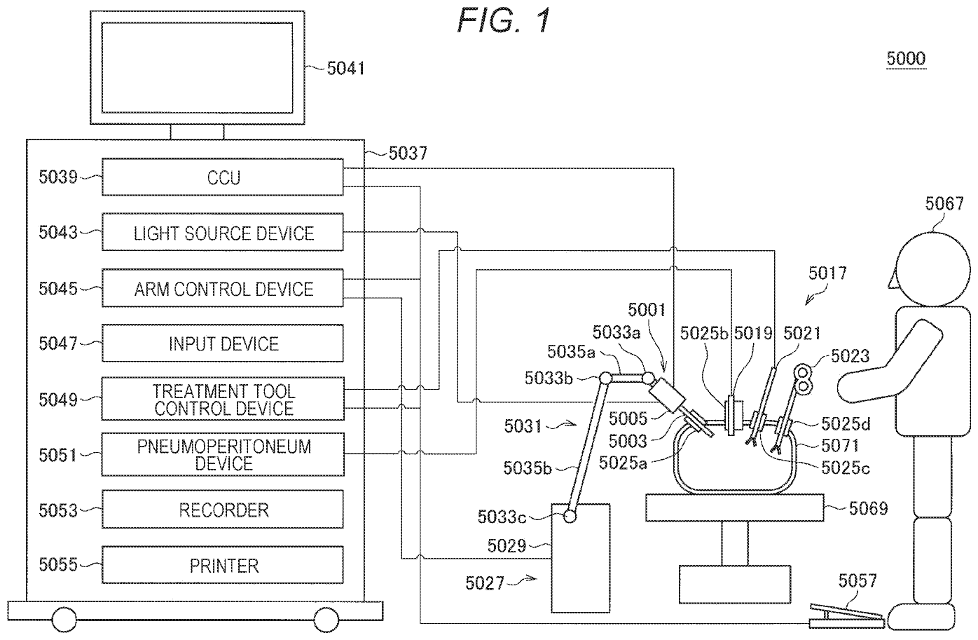

FIG. 1 is a diagram illustrating an example of a schematic configuration of an endoscopic surgical system 5000 to which the technology according to the present disclosure is applicable. FIG. 1 illustrates a state in which an operator (surgeon) 5067 is performing an operation on a patient 5071 on a patient bed 5069, using the endoscopic surgical system 5000. As illustrated, the endoscopic surgical system 5000 includes an endoscope 5001, other surgical tools 5017, a support arm device 5027 that supports the endoscope 5001, and a cart 5037 in which various devices for endoscopic surgery are mounted.

In an endoscopic surgery, a plurality of cylindrical puncture instruments called trocars 5025a to 5025d is punctured into an abdominal wall instead of cutting the abdominal wall and opening the abdomen. Then, a lens barrel 5003 of the endoscope 5001 and other surgical tools 5017 are inserted into a body cavity of the patient 5071 through the trocars 5025a to 5025d. In the illustrated example, as the other surgical tools 5017, a pneumoperitoneum tube 5019, an energy treatment tool 5021, and a forceps 5023 are inserted into the body cavity of the patient 5071. Furthermore, the energy treatment tool 5021 is a treatment tool for performing incision and detachment of tissue, sealing of a blood vessel, and the like with a high-frequency current or an ultrasonic vibration. Note that the illustrated surgical tools 5017 are mere examples, and various kinds of surgical tools typically used in endoscopic surgery such as tweezers and a retractor may be used as the surgical tool 5017.

An image of an operation site in the body cavity of the patient 5071 captured by the endoscope 5001 is displayed on a display device 5041. The operator 5067 performs treatment such as removal of an affected part, for example, using the energy treatment tool 5021 and the forceps 5023 while viewing the image of the operation site displayed on the display device 5041 in real time. Note that the pneumoperitoneum tube 5019, the energy treatment tool 5021, and the forceps 5023 are supported by the operator 5067, an assistant, or the like during surgery, although illustration is omitted.

(Support Arm Device)

The support arm device 5027 includes an arm unit 5031 extending from a base unit 5029. In the illustrated example, the arm unit 5031 includes joint units 5033a, 5033b, and 5033c, and links 5035a and 5035b, and is driven under the control of an arm control device 5045. The endoscope 5001 is supported by the arm unit 5031, and the position and posture of the endoscope 5001 are controlled. With the control, stable fixation of the position of the endoscope 5001 can be realized.

(Endoscope)

The endoscope 5001 includes the lens barrel 5003 and a camera head 5005. A region having a predetermined length from a distal end of the lens barrel 5003 is inserted into the body cavity of the patient 5071. The camera head 5005 is connected to a proximal end of the lens barrel 5003. In the illustrated example, the endoscope 5001 configured as a so-called hard endoscope including the hard lens barrel 5003 is illustrated. However, the endoscope 5001 may be configured as a so-called soft endoscope including the soft lens barrel 5003.

An opening portion in which an object lens is fit is provided in the distal end of the lens barrel 5003. A light source device 5043 is connected to the endoscope 5001, and light generated by the light source device 5043 is guided to the distal end of the lens barrel 5003 by a light guide extending inside the lens barrel 5003 and an observation target in the body cavity of the patient 5071 is irradiated with the light through the object lens. Note that the endoscope 5001 may be a forward-viewing endoscope, an oblique-viewing endoscope, or a side-viewing endoscope.

An optical system and an imaging element are provided inside the camera head 5005, and reflected light (observation light) from the observation target is condensed to the imaging element by the optical system. The observation light is photoelectrically converted by the imaging element, and an electrical signal corresponding to the observation light, that is, an image signal corresponding to an observed image is generated. The image signal is transmitted to a camera control unit (CCU) 5039 as raw data. Note that the camera head 5005 has a function to adjust magnification and a focal length by appropriately driving the optical system.

Note that a plurality of the imaging elements may be provided in the camera head 5005 to support three-dimensional (3D) display, and the like, for example. In this case, a plurality of relay optical systems is provided inside the lens barrel 5003 to guide the observation light to each of the plurality of imaging elements.

(Various Devices Mounted in Cart)

The CCU 5039 includes a central processing unit (CPU), a graphics processing unit (GPU), and the like, and centrally controls the operation of the endoscope 5001 and the display device 5041. Specifically, the CCU 5039 receives the image signal from the camera head 5005, and applies various types of image processing for displaying an image based on the image signal, such as developing processing (demosaicing processing), for example, to the image signal. The CCU 5039 provides the image signal to which the image processing has been applied to the display device 5041. Furthermore, the CCU 5039 transmits a control signal to the camera head 5005 to control its driving. The control signal may include information regarding imaging conditions such as the magnification and focal length.

The display device 5041 displays an image based on the image signal to which the image processing has been applied by the CCU 5039, under the control of the CCU 5039. In a case where the endoscope 5001 supports high-resolution capturing such as 4K (horizontal pixel number 3840×vertical pixel number 2160) or 8K (horizontal pixel number 7680×vertical pixel number 4320), and/or in a case where the endoscope 5001 supports 3D display, for example, the display device 5041, which can perform high-resolution display and/or 3D display, can be used corresponding to each case. In a case where the endoscope 5001 supports the high-resolution capturing such as 4K or 8K, a greater sense of immersion can be obtained by use of the display device 5041 with the size of 55 inches or more. Furthermore, a plurality of display devices 5041 having different resolutions and sizes may be provided depending on the application.

The light source device 5043 includes a light source such as a light emitting diode (LED) for example, and supplies irradiation light to the endoscope 5001 in capturing an operation portion.

The arm control device 5045 includes a processor such as a CPU, and is operated according to a predetermined program, thereby to control driving of the arm unit 5031 of the support arm device 5027 according to a predetermined control method.

An input device 5047 is an input interface for the endoscopic surgical system 5000. The user can input various types of information and instructions to the endoscopic surgical system 5000 through the input device 5047. For example, the user inputs various types of information regarding surgery, such as patient's physical information and information of an operative procedure of the surgery, through the input device 5047. Furthermore, for example, the user inputs an instruction to drive the arm unit 5031, an instruction to change the imaging conditions (such as the type of the irradiation light, the magnification, and the focal length) of the endoscope 5001, an instruction to drive the energy treatment tool 5021, or the like through the input device 5047.

The type of the input device 5047 is not limited, and the input device 5047 may be one of various known input devices. For example, a mouse, a keyboard, a touch panel, a switch, a foot switch 5057, and/or a lever can be applied to the input device 5047. In a case where a touch panel is used as the input device 5047, the touch panel may be provided on a display surface of the display device 5041.

Alternatively, the input device 5047 is a device worn by the user, such as a glass-type wearable device or a head mounted display (HMD), for example, and various inputs are performed according to a gesture or a line of sight of the user detected by the device. Furthermore, the input device 5047 includes a camera capable of detecting a movement of the user, and various inputs are performed according to a gesture or a line of sight of the user detected from a video captured by the camera. Moreover, the input device 5047 includes a microphone capable of collecting a voice of the user, and various inputs are performed by an audio through the microphone. In this way, the input device 5047 is configured to be able to input various types of information in a non-contact manner, whereby the user (for example, the operator 5067) in particular belonging to a clean area can operate a device belonging to a filthy area in a non-contact manner. Furthermore, since the user can operate the device without releasing his/her hand from the possessed surgical tool, the user's convenience is improved.

A treatment tool control device 5049 controls driving of the energy treatment tool 5021 for cauterization and incision of tissue, sealing of a blood vessel, and the like. A pneumoperitoneum device 5051 sends a gas into the body cavity of the patient 5071 through the pneumoperitoneum tube 5019 to expand the body cavity for the purpose of securing a field of view by the endoscope 5001 and a work space for the operator. A recorder 5053 is a device that can record various types of information regarding the surgery. A printer 5055 is a device that can print the various types of information regarding the surgery in various formats such as a text, an image, or a graph.

Hereinafter, a particularly characteristic configuration in the endoscopic surgical system 5000 will be further described in detail.

(Support Arm Device)

The support arm device 5027 includes the base unit 5029 as a base and the arm unit 5031 extending from the base unit 5029. In the illustrated example, the arm unit 5031 includes the plurality of joint units 5033a, 5033b, and 5033c and the plurality of links 5035a and 5035b connected by the joint unit 5033b, but FIG. 1 illustrates the configuration of the arm unit 5031 in a simplified manner for simplification. In reality, the shapes, the number, and the arrangement of the joint units 5033a to 5033c and the links 5035a and 5035b, the directions of rotation axes of the joint units 5033a to 5033c, and the like can be appropriately set so that the arm unit 5031 has a desired degree of freedom. For example, the arm unit 5031 can be favorably configured to have six degrees of freedom or more. With the configuration, the endoscope 5001 can be freely moved within a movable range of the arm unit 5031. Therefore, the lens barrel 5003 of the endoscope 5001 can be inserted from a desired direction into the body cavity of the patient 5071.

Actuators are provided in the joint units 5033a to 5033c, and the joint units 5033a to 5033c are configured to be rotatable around a predetermined rotation axis by driving of the actuators. The driving of the actuators is controlled by the arm control device 5045, whereby rotation angles of the joint units 5033a to 5033c are controlled and driving of the arm unit 5031 is controlled. With the control, control of the position and posture of the endoscope 5001 can be realized. At this time, the arm control device 5045 can control the driving of the arm unit 5031 by various known control methods such as force control or position control.

For example, the driving of the arm unit 5031 may be appropriately controlled by the arm control device 5045 according to an operation input, and the position and posture of the endoscope 5001 may be controlled, by an appropriate operation input by the operator 5067 via the input device 5047 (including the foot switch 5057). With the control, the endoscope 5001 at the distal end of the arm unit 5031 can be moved from an arbitrary position to an arbitrary position, and then can be fixedly supported at the position after the movement. Note that the arm unit 5031 may be operated by a so-called master-slave system. In this case, the arm unit 5031 can be remotely operated by the user via the input device 5047 installed at a place distant from an operating room.

Furthermore, in a case where the force control is applied, the arm control device 5045 may perform so-called power assist control in which the arm control device 5045 receives an external force from the user and drives the actuators of the joint units 5033a to 5033c so that the arm unit 5031 is smoothly moved according to the external force. With the control, the user can move the arm unit 5031 with a relatively light force when moving the arm unit 5031 while being in direct contact with the arm unit 5031. Accordingly, the user can more intuitively move the endoscope 5001 with a simpler operation, and the user's convenience can be improved.

Here, in endoscopic surgery, the endoscope 5001 has been generally supported by a surgeon called scopist. In contrast, by use of the support arm device 5027, the position of the endoscope 5001 can be reliably fixed without manual operation, and thus an image of the operation site can be stably obtained and the surgery can be smoothly performed.

Note that the arm control device 5045 is not necessarily provided in the cart 5037. Furthermore, the arm control device 5045 is not necessarily one device. For example, the arm control device 5045 may be provided in each of the joint units 5033a to 5033c of the arm unit 5031 of the support arm device 5027, and the drive control of the arm unit 5031 may be realized by mutual cooperation of the plurality of arm control devices 5045.

(Light Source Device)

The light source device 5043 supplies irradiation light, which is used in capturing an operation site, to the endoscope 5001. The light source device 5043 includes, for example, an LED, a laser light source, or a white light source configured by a combination thereof. In a case where the white light source is configured by a combination of RGB laser light sources, output intensity and output timing of the respective colors (wavelengths) can be controlled with high accuracy. Therefore, white balance of a captured image can be adjusted in the light source device 5043. Further, in this case, the observation target is irradiated with the laser light from each of the RGB laser light sources in a time division manner, and the driving of the imaging element of the camera head 5005 is controlled in synchronization with the irradiation timing, so that images respectively corresponding to RGB can be captured in a time division manner. According to the method, a color image can be obtained without providing a color filter to the imaging element.

Furthermore, driving of the light source device 5043 may be controlled to change intensity of light to be output every predetermined time. The driving of the imaging element of the camera head 5005 is controlled in synchronization with change timing of the intensity of light, and images are acquired in a time division manner and are synthesized, whereby a high-dynamic range image without clipped blacks and flared highlights can be generated.

Further, the light source device 5043 may be configured to be able to supply light in a predetermined wavelength band corresponding to special light observation. In the special light observation, for example, so-called narrow band imaging is performed by radiating light in a narrower band than the irradiation light (in other words, white light) at the time of normal observation, using wavelength dependence of absorption of light in a body tissue, to capture a predetermined tissue such as a blood vessel in a mucosal surface layer at high contrast. Alternatively, in the special light observation, fluorescence observation to obtain an image by fluorescence generated by radiation of exciting light may be performed. In the fluorescence observation, irradiating the body tissue with exciting light to observe fluorescence from the body tissue (self-fluorescence observation), injecting a reagent such as indocyanine green (ICG) into the body tissue and irradiating the body tissue with exciting light corresponding to a fluorescence wavelength of the reagent to obtain a fluorescence image, or the like can be performed. The light source device 5043 can be configured to be able to supply narrow-band light and/or exciting light corresponding to such special light observation.

(Camera Head and CCU)

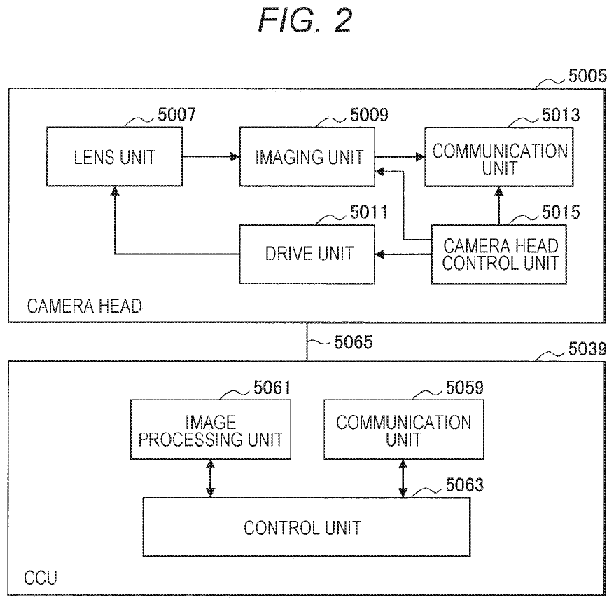

Functions of the camera head 5005 and the CCU 5039 of the endoscope 5001 will be described in more detail with reference to FIG. 2. FIG. 2 is a block diagram illustrating an example of functional configurations of the camera head 5005 and the CCU 5039 illustrated in FIG. 1.

Referring to FIG. 2, the camera head 5005 includes a lens unit 5007, an imaging unit 5009, a drive unit 5011, a communication unit 5013, and a camera head control unit 5015 as its functions. Furthermore, the CCU 5039 includes a communication unit 5059, an image processing unit 5061, and a control unit 5063 as its functions. The camera head 5005 and the CCU 5039 are communicatively connected with each other by a transmission cable 5065.

First, a functional configuration of the camera head 5005 will be described. The lens unit 5007 is an optical system provided in a connection portion between the lens unit 5007 and the lens barrel 5003. Observation light taken through the distal end of the lens barrel 5003 is guided to the camera head 5005 and enters the lens unit 5007. The lens unit 5007 is configured by a combination of a plurality of lenses including a zoom lens and a focus lens. Optical characteristics of the lens unit 5007 are adjusted to condense the observation light on a light receiving surface of an imaging element of the imaging unit 5009. Furthermore, the zoom lens and the focus lens are configured to have their positions on the optical axis movable for adjustment of the magnification and focal point of the captured image.

The imaging unit 5009 includes an imaging element, and is disposed at a rear stage of the lens unit 5007. The observation light having passed through the lens unit 5007 is focused on the light receiving surface of the imaging element, and an image signal corresponding to the observed image is generated by photoelectric conversion. The image signal generated by the imaging unit 5009 is provided to the communication unit 5013.

As the imaging element constituting the imaging unit 5009, for example, a complementary metal oxide semiconductor (CMOS)-type image sensor having Bayer arrangement and capable of color capturing is used. Note that, as the imaging element, for example, an imaging element that can capture a high-resolution image of 4K or more may be used. By obtainment of the image of the operation site with high resolution, the operator 5067 can grasp the state of the operation site in more detail and can more smoothly advance the surgery.

Furthermore, the imaging element constituting the imaging unit 5009 includes a pair of imaging elements for respectively obtaining image signals for right eye and for left eye corresponding to 3D display. With the 3D display, the operator 5067 can more accurately grasp the depth of biological tissue in the operation site. Note that, in a case where the imaging unit 5009 is configured as a multi-plate imaging unit, a plurality of systems of the lens units 5007 is provided corresponding to the imaging elements.

Furthermore, the imaging unit 5009 may not be necessarily provided in the camera head 5005. For example, the imaging unit 5009 may be provided immediately after the object lens inside the lens barrel 5003.

The drive unit 5011 includes an actuator, and moves the zoom lens and the focus lens of the lens unit 5007 by a predetermined distance along an optical axis by the control of the camera head control unit 5015. With the movement, the magnification and focal point of the captured image by the imaging unit 5009 can be appropriately adjusted.

The communication unit 5013 includes a communication device for transmitting or receiving various types of information to or from the CCU 5039. The communication unit 5013 transmits the image signal obtained from the imaging unit 5009 to the CCU 5039 through the transmission cable 5065 as raw data. At this time, to display the captured image of the operation site with low latency, the image signal is favorably transmitted by optical communication. This is because, in surgery, the operator 5067 performs surgery while observing the state of the affected part with the captured image, and thus display of a moving image of the operation site in as real time as possible is demanded for more safe and reliable surgery. In the case of the optical communication, a photoelectric conversion module that converts an electrical signal into an optical signal is provided in the communication unit 5013. The image signal is converted into the optical signal by the photoelectric conversion module, and is then transmitted to the CCU 5039 via the transmission cable 5065.

Furthermore, the communication unit 5013 receives a control signal for controlling driving of the camera head 5005 from the CCU 5039. The control signal includes information regarding the imaging conditions such as information for specifying a frame rate of the captured image, information for specifying an exposure value at the time of imaging, and/or information for specifying the magnification and the focal point of the captured image, for example. The communication unit 5013 provides the received control signal to the camera head control unit 5015. Note that the control signal from that CCU 5039 may also be transmitted by the optical communication. In this case, the communication unit 5013 is provided with a photoelectric conversion module that converts an optical signal into an electrical signal, and the control signal is converted into an electrical signal by the photoelectric conversion module and is then provided to the camera head control unit 5015.

Note that the imaging conditions such as the frame rate, exposure value, magnification, and focal point are automatically set by the control unit 5063 of the CCU 5039 on the basis of the acquired image signal. That is, a so-called auto exposure (AE) function, an auto focus (AF) function, and an auto white balance (AWB) function are incorporated in the endoscope 5001.

The camera head control unit 5015 controls the driving of the camera head 5005 on the basis of the control signal received from the CCU 5039 through the communication unit 5013. For example, the camera head control unit 5015 controls driving of the imaging element of the imaging unit 5009 on the basis of the information for specifying the frame rate of the captured image and/or the information for specifying exposure at the time of imaging. Furthermore, for example, the camera head control unit 5015 appropriately moves the zoom lens and the focus lens of the lens unit 5007 via the drive unit 5011 on the basis of the information for specifying the magnification and focal point of the captured image. The camera head control unit 5015 may further have a function to store information for identifying the lens barrel 5003 and the camera head 5005.

Note that the configuration of the lens unit 5007, the imaging unit 5009, and the like is arranged in a hermetically sealed structure having high airtightness and waterproofness, whereby the camera head 5005 can have resistance to autoclave sterilization processing.

Next, a functional configuration of the CCU 5039 will be described. The communication unit 5059 includes a communication device for transmitting or receiving various types of information to or from the camera head 5005. The communication unit 5059 receives the image signal transmitted from the camera head 5005 through the transmission cable 5065. At this time, as described above, the image signal can be favorably transmitted by the optical communication. In this case, the communication unit 5059 is provided with a photoelectric conversion module that converts an optical signal into an electrical signal, corresponding to the optical communication. The communication unit 5059 provides the image signal converted into the electrical signal to the image processing unit 5061.

Furthermore, the communication unit 5059 transmits a control signal for controlling driving of the camera head 5005 to the camera head 5005. The control signal may also be transmitted by the optical communication.

The image processing unit 5061 applies various types of image processing to the image signal as raw data transmitted from the camera head 5005. The image processing include various types of known signal processing such as development processing, high image quality processing (such as band enhancement processing, super resolution processing, noise reduction (NR) processing, and/or camera shake correction processing), and/or enlargement processing (electronic zoom processing), for example. Furthermore, the image processing unit 5061 performs wave detection processing for image signals for performing AE, AF, and AWB.

The image processing unit 5061 is configured by a processor such as a CPU or a GPU, and the processor is operated according to a predetermined program, whereby the above-described image processing and wave detection processing can be performed. Note that in a case where the image processing unit 5061 includes a plurality of GPUs, the image processing unit 5061 appropriately divides the information regarding the image signal and performs the image processing in parallel by the plurality of GPUs.

The control unit 5063 performs various types of control related to imaging of the operation site by the endoscope 5001 and display of the captured image. For example, the control unit 5063 generates a control signal for controlling driving of the camera head 5005. At this time, in a case where the imaging conditions are input by the user, the control unit 5063 generates the control signal on the basis of the input by the user. Alternatively, in a case where the AE function, the AF function, and the AWB function are incorporated in the endoscope 5001, the control unit 5063 appropriately calculates optimum exposure value, focal length, and white balance according to a result of the wave detection processing by the image processing unit 5061, and generates the control signal.

Furthermore, the control unit 5063 displays the image of the operation site on the display device 5041 on the basis of the image signal to which the image processing has been applied by the image processing unit 5061. At this time, the control unit 5063 recognizes various objects in the image of the operation site, using various image recognition technologies. For example, the control unit 5063 can recognize a surgical instrument such as forceps, a specific living body portion, blood, mist at the time of use of the energy treatment tool 5021, or the like, by detecting a shape of an edge, a color or the like of an object included in the operation site image. The control unit 5063 superimposes and displays various types of surgery support information on the image of the operation site, in displaying the image of the operation site on the display device 5041 using the result of recognition. The surgery support information is superimposed, displayed, and presented to the operator 5067, so that the surgery can be more safely and reliably advanced.

The transmission cable 5065 that connects the camera head 5005 and the CCU 5039 is an electrical signal cable supporting communication of electrical signals, an optical fiber supporting optical communication, or a composite cable thereof.

Here, in the illustrated example, the communication has been performed in a wired manner using the transmission cable 5065. However, the communication between the camera head 5005 and the CCU 5039 may be wirelessly performed. In a case where the communication between the camera head 5005 and the CCU 5039 is wirelessly performed, it is unnecessary to lay the transmission cable 5065 in the operating room. Therefore, the situation in which movement of medical staffs in the operating room is hindered by the transmission cable 5065 can be eliminated.

An example of an endoscopic surgical system 5000 to which the technology according to the present disclosure is applicable has been described. Note that, here, the endoscopic surgical system 5000 has been described as an example. However, a system to which the technology according to the present disclosure is applicable is not limited to this example. For example, the technique according to the present disclosure may be applied to a flexible endoscopic system for examination or a microsurgical system.

### 2. SPECIFIC CONFIGURATION EXAMPLE OF SUPPORT ARM DEVICE

Next, a specific configuration example of a support arm device according to the embodiment of the present disclosure will be described in detail. The support arm device described below is an example configured as a support arm device that supports an endoscope at a distal end of an arm unit. However, the present embodiment is not limited to the example. Furthermore, in a case where the support arm device according to the embodiment of the present disclosure is applied to the medical field, the support arm device according to the embodiment of the present disclosure can function as a medical support arm device.

<2-1. Appearance of Support Arm Device>

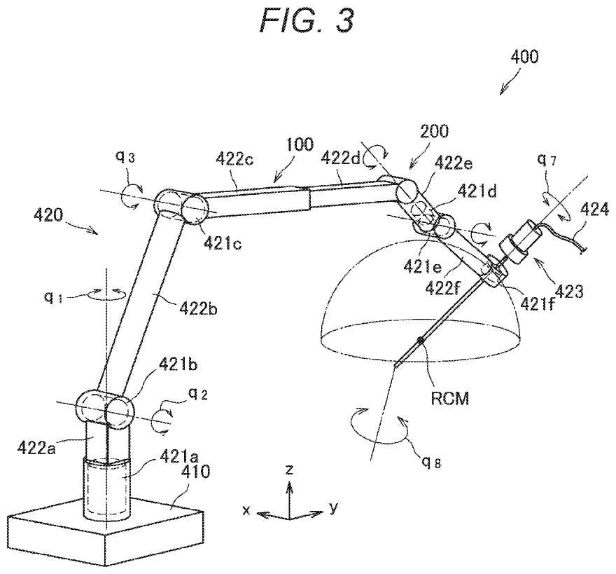

First, a schematic configuration of a support arm device 400 according to the present embodiment will be described with reference to FIG. 3. FIG. 3 is a schematic view illustrating an appearance of the support arm device 400 according to the present embodiment.

The support arm device 400 according to the present embodiment includes a base unit 410 and an arm unit 420. The base unit 410 is a base of the support arm device 400, and the arm unit 420 is extended from the base unit 410. Furthermore, although not illustrated in FIG. 3, a control unit that integrally controls the support arm device 400 may be provided in the base unit 410, and driving of the arm unit 420 may be controlled by the control unit. The control unit includes various signal processing circuits, such as a CPU and a DSP, for example.

The arm unit 420 includes a plurality of active joint units 421a to 421f, a plurality of links 422a to 422f, and an endoscope device 423 as a distal end unit provided at a distal end of the arm unit 420.

The links 422a to 422f are substantially rod-like members. One end of the link 422a is connected to the base unit 410 via the active joint unit 421a, the other end of the link 422a is connected to one end of the link 422b via the active joint unit 421b, and the other end of the link 422b is connected to one end of the link 422c via the active joint unit 421c. The other end of the link 422c is connected to the link 422d via a passive slide mechanism 100, and the other end of the link 422d is connected to one end of the link 422e via a passive joint unit 200. The other end of the link 422e is connected to one end of the link 422f via the active joint units 421d and 421 e. The endoscope device 423 is connected to the distal end of the arm unit 420, in other words, the other end of the link 422f, via the active joint unit 421f. The respective ends of the plurality of links 422a to 422f are connected one another by the active joint units 421a to 421f, the passive slide mechanism 100, and the passive joint unit 200 with the base unit 410 as a fulcrum, as described above, so that an arm shape extended from the base unit 410 is configured.

Actuators provided in the respective active joint units 421a to 421 f of the arm unit 420 are driven and controlled, so that the position and posture of the endoscope device 423 are controlled. In the present embodiment, the endoscope device 423 has a distal end enter a body cavity of a patient, which is an operation site, and captures a partial region of the operation site. Note that the distal end unit provided at the distal end of the arm unit 420 is not limited to the endoscope device 423, and various medical instruments may be connected to the distal end of the arm unit 420 as the distal end units. Thus, the support arm device 400 according to the present embodiment is configured as a medical support arm device provided with a medical instrument.

Here, hereinafter, the support arm device 400 will be described by defining coordinate axes as illustrated in FIG. 3. Furthermore, an up-down direction, a front-back direction, and a right-left direction will be defined in accordance with the coordinate axes. In other words, the up-down direction with respect to the base unit 410 installed on a floor is defined as a z-axis direction and the up-down direction Furthermore, a direction orthogonal to the z axis and in which the arm unit 420 is extended from the base unit 410 (in other words, a direction in which the endoscope device 423 is located with respect to the base unit 410) is defined as a y-axis direction and the front-back direction. Moreover, a direction orthogonal to the y axis and the z axis is defined as an x-axis direction and the right-left direction.

The active joint units 421a to 421f rotatably connect the links to one another. The active joint units 421a to 421f include actuators, and have a rotation mechanism that is rotationally driven about a predetermined rotation axis by driving of the actuators. By controlling rotational driving of each of the active joint units 421a to 421f, driving of the arm unit 420 such as extending or contracting (folding) of the arm unit 420 can be controlled. Here, the driving of the active joint units 421a to 421f can be controlled by, for example, known whole body coordination control and ideal joint control. As described above, since the active joint units 421a to 421f have the rotation mechanism, in the following description, the drive control of the active joint units 421a to 421 f specifically means control of rotation angles and/or generated torque (torque generated by the active joint units 421a to 421f) of the active joint units 421a to 421f.

The passive slide mechanism 100 is an aspect of a passive form change mechanism, and connects the link 422c and the link 422d to be able to move forward and backward along a predetermined direction. For example, the passive slide mechanism 100 may connect the link 422c and the link 422d in a linearly movable manner. However, the forward/backward motion of the link 422c and the link 422d is not limited to the linear motion, and may be forward/backward motion in a direction of forming an arc. The passive slide mechanism 100 is operated in the forward/backward motion by a user, for example, and makes a distance between the active joint unit 421c on the one end side of the link 422c and the passive joint unit 200 variable. Thereby, the entire form of the arm unit 420 can change.

The passive joint unit 200 is one aspect of the passive form change mechanism, and rotatably connects the link 422d and the link 422e to each other. The passive joint unit 200 is rotatably operated by the user, for example, and makes an angle made by the link 422d and the link 422e variable. Thereby, the entire form of the arm unit 420 can change.

Note that, in the present specification, a “posture of the arm unit” refers to a state of the arm unit changeable by the drive control of the actuators provided in the active joint units 421a to 421f by the control unit in a state where the distance between active joint units adjacent across one or a plurality of links is constant. Furthermore, a “form of the arm unit” refers to a state of the arm unit changeable as the distance between active joint units adjacent across a link or an angle between links connecting adjacent active joint units changes with the operation of the passive form change mechanism.

The support arm device 400 according to the present embodiment includes the six active joint units 421a to 421f and realizes six degrees of freedom with respect to the driving of the arm unit 420. That is, while the drive control of the support arm device 400 is realized by the drive control of the six active joint units 421a to 421f by the control unit, the passive slide mechanism 100 and the passive joint unit 200 are not the targets of the drive control by the control unit.

Specifically, as illustrated in FIG. 3, the active joint units 421a, 421d, and 421f are provided to have long axis directions of the connected links 422a and 422e and a capture direction of the connected endoscope device 423 as rotation axis directions. The active joint units 421b, 421c, and 421e are provided to have the x-axis direction that is a direction in which connection angles of the connected links 422a to 422c, 422e, and 422f and the connected endoscope device 423 are changed in a y-z plane (a plane defined by the y axis and the z axis) as rotation axis directions. As described above, in the present embodiment, the active joint units 421a, 421d, and 421f have a function to perform so-called yawing, and the active joint units 421b, 421c, and 421e have a function to perform so-called pitching.

With the above configuration of the arm unit 420, the support arm device 400X) according to the present embodiment realizes the six degrees of freedom with respect to the driving of the arm unit 420, whereby freely moving the endoscope device 423 within the movable range of the arm unit 420. FIG. 3 illustrates a hemisphere as an example of a movable range of the endoscope device 423. In a case where a central point RCM (remote motion center) of the hemisphere is a capture center of the operation site captured by the endoscope device 423, the operation site can be captured from various angles by moving the endoscope device 423 on a spherical surface of the hemisphere in a state where the capture center of the endoscope device 423 is fixed to the central point of the hemisphere.

The schematic configuration of the support arm device 400 according to the present embodiment has been described above. Next, the whole body coordination control and the ideal joint control for controlling the driving of the arm unit 420, in other words, the driving of the joint units 421a to 421f in the support arm device 400 according to the present embodiment will be described.

<2-2. Generalized Inverse Dynamics>

Next, an overview of generalized inverse dynamics used for the whole body coordination control of the support arm device 400 in the present embodiment will be described.

The generalized inverse dynamics is basic operation in the whole body coordination control of a multilink structure configured by connecting a plurality of links by a plurality of joint units (for example, the arm unit 420 illustrated in FIG. 2 in the present embodiment), for converting motion purposes regarding various dimensions in various operation spaces into torque to be caused in the plurality of joint units in consideration of various constraint conditions.

The operation space is an important concept in force control of a robot device. The operation space is a space for describing a relationship between force acting on the multilink structure and acceleration of the multilink structure. When the drive control of the multilink structure is performed not by position control but by force control, the concept of the operation space is required in a case of using a contact between the multilink structure and an environment as a constraint condition. The operation space is, for example, a joint space, a Cartesian space, a momentum space, or the like, which is a space to which the multilink structure belongs.

The motion purpose represents a target value in the drive control of the multilink structure, and is, for example, a target value of a position, a speed, an acceleration, a force, an impedance, or the like of the multilink structure to be achieved by the drive control.

The constraint condition is a constraint condition regarding the position, speed, acceleration, force, or the like of the multilink structure, which is determined according to a shape or a structure of the multilink structure, an environment around the multilink structure, settings by the user, and the like. For example, the constraint condition includes information regarding a generated force, a priority, presence/absence of a non-drive joint, a vertical reaction force, a friction weight, a support polygon, and the like.

In the generalized dynamics, to establish both stability of numerical calculation and real time processing efficiency, an arithmetic algorithm includes a virtual force determination process (virtual force calculation processing) as a first stage and a real force conversion process (real force calculation processing) as a second stage. In the virtual force calculation processing as the first stage, a virtual force that is a virtual force required for achievement of each motion purpose and acting on the operation space is determined while considering the priority of the motion purpose and a maximum value of the virtual force. In the real force calculation processing as the second stage, the above-obtained virtual force is converted into a real force realizable in the actual configuration of the multilink structure, such as a joint force or an external force, while considering the constraints regarding the non-drive joint, the vertical reaction force, the friction weight, the support polygon, and the like. Hereinafter, the virtual force calculation processing and the real force calculation processing will be described in detail. Note that, in the description of the virtual force calculation processing and the real force calculation processing below and the real force calculation processing to be described below, description may be performed using the configuration of the arm unit 420 of the support arm device 400 according to the present embodiment illustrated in FIG. 3 as a specific example, in order to facilitate understanding.

(2-2-1. Virtual Force Calculation Processing)

A vector configured by a certain physical quantity at each joint unit of the multilink structure is called generalized variable q (also referred to as a joint value q or a joint space q). An operation space x is defined by the following expression (1) using a time derivative value of the generalized variable q and the Jacobian J.

[Math. 1]

\dot{x}=J\dot{q} (1)

In the present embodiment, for example, q is a rotation angle of the joint units 421a to 421f of the arm unit 420. An equation of motion regarding the operation space x is described by the following expression (2).

[Math. 2]

\ddot{x}=Λ⁻¹f+c (2)

Here, f represents a force acting on the operation space x. Furthermore, Λ⁻¹ is an operation space inertia inverse matrix, and c is called operation space bias acceleration, which are respectively expressed by the following expressions (3) and (4).

[Math. 3]

Λ⁻¹=JH⁻¹J^{T} (3)

c=JH⁻¹(τ−b)+\dot{J}ġ (4)

Note that H represents a joint space inertia matrix, τ represents a joint force corresponding to the joint value q (for example, the generated torque at the joint units 421a to 421f), and b represents gravity, a Coriolis force, and a centrifugal force.

In the generalized inverse dynamics, it is known that the motion purpose of the position and speed regarding the operation space x can be expressed as an acceleration of the operation space x. At this time, the virtual force f to act on the operation space x to realize an operation space acceleration that is a target value given as the motion purpose can be obtained by solving a kind of linear complementary problem (LCP) as in the expression (5) below according to the above expression (1).

$$\begin{matrix}[ Math . 4 ] & \\ w + \ddot{x} = {Λ}^{- 1} {f}_{v} + c & ( 5 ) \\ s . t . \{ \begin{matrix}( ( {w}_{i} < 0 ) ⋂ ( {f}_{{v}_{i}} = {U}_{i} ) ) ⋃ \\ ( ( {w}_{i} > 0 ) ⋂ ( {f}_{{v}_{i}} = {L}_{i} ) ) ⋃ \\ ( ( {w}_{i} = 0 ) ⋂ ( {L}_{i} < {f}_{{v}_{i}} < {U}_{i} ) )\end{matrix} & \end{matrix}$$

Here, Lᵢ and Uᵢ respectively represent a negative lower limit value (including −∞) of an i-th component of fᵥ and a positive upper limit value (including +∞) of the i-th component of fᵥ. The above LCP can be solved using, for example, an iterative method, a pivot method, a method applying robust acceleration control, or the like.

Note that the operation space inertia inverse matrix Λ⁻¹ and the bias acceleration c have a large calculation cost when calculated according to the expressions (3) and (4) that are defining expressions. Therefore, a method of calculating the processing of calculating the operation space inertia inverse matrix Λ⁻¹ at a high speed by applying a quasi-dynamics operation (FWD) for obtaining a generalized acceleration (joint acceleration) from the generalized force (joint force r) of the multilink structure has been proposed. Specifically, the operation space inertia inverse matrix Λ⁻¹ and the bias acceleration c can be obtained from information regarding forces acting on the multilink structure (for example, the joint units 421a to 421f of the arm unit 420), such as the joint space q, the joint force τ, and the gravity g by using the forward dynamics operation FWD. The operation space inertia inverse matrix Λ⁻¹ can be calculated with a calculation amount of O (N) with respect to the number (N) of the joint units by applying the forward dynamics operation FWD regarding the operation space.

Here, as a setting example of the motion purpose, a condition for achieving the target value (expressed by adding a superscript bar to second-order differentiation of x) of the operation space acceleration with a virtual force fᵥᵢ equal to or smaller than an absolute value Fᵢ can be expressed by the following expression (6).

[Math. 5]

Lᵢ=−Fᵢ,

Uᵢ=Fᵢ,

\ddot{x}ᵢ=\ddot{x̅}ᵢ (6)

Furthermore, as described above, the motion purpose regarding the position and speed of the operation space x can be expressed as the target value of the operation space acceleration, and is specifically expressed by the following expression (7) (the target value of the position and speed of the operation space x is expressed by x and adding the superscript bar to first-order differentiation of x).

[Math. 6]

\ddot{x̅}ᵢ=Kₚ(x̅ᵢ−xᵢ)+Kᵥ(\dot{x̅}ᵢ−\dot{x}ᵢ) (7)

In addition, by use of a concept of decomposition operation space, the motion purpose regarding an operation space (momentum, Cartesian relative coordinates, interlocking joint, or the like) expressed by a linear sum of other operation spaces. Note that it is necessary to give priority to competing motion purposes. The above LCP can be solved for each priority in ascending order from a low priority, and the virtual force obtained by the LCP in the previous stage can be made to act as a known external force of the LCP in the next stage.

(2-2-2. Real Force Calculation Processing)

In the real force calculation processing as the second stage of the generalized inverse dynamics, processing of replacing the virtual force fᵥ obtained in the above (2-2-1. Virtual Force Determination Process) with real joint force and external force is performed. A condition for realizing the generalized force τᵥ=Jᵥ^{T}fᵥ generated in the joint unit by the virtual force with generated torque τₐ and an external force fₑ is expressed by the following expression (8).

$$\begin{matrix}[ Math . 7 ] & \\ [ \begin{matrix}{J}_{vu}^{T} \\ {J}_{v a}^{T}\end{matrix} ] ( {f}_{v} - Δ {f}_{v} ) = [ \begin{matrix}{J}_{eu}^{T} \\ {J}_{ea}^{T}\end{matrix} ] {f}_{e} + [ \begin{matrix}0 \\ {τ}_{a}\end{matrix} ] & ( 8 )\end{matrix}$$

Here, the suffix a represents a set of drive joint units (drive joint set), and the suffix u represents a set of non-drive joint units (non-drive joint set). In other words, the upper part of the above expression (8) represents balance of the forces of the space (non-drive joint space) by the non-drive joint units, and the lower part represents balance of the forces of the space (drive joint space) by the drive joint units. Jᵥᵤ and Jᵥₐ are respectively a non-drive joint component and a drive joint component of the Jacobian regarding the operation space where the virtual force fᵥ acts. Jₑᵤ and Jₑₐ are a non-drive joint component and a drive joint component of the Jacobian regarding the operation space where the external force fₑ acts. Δfᵥ represents an unrealizable component with the real force, of the virtual force fᵥ.

The upper part of the expression (8) is undefined. For example, f_{f} and Δfᵥ can be obtained by solving a quadratic programming problem (QP) as described in the following expression (9).

$$\begin{matrix}[ Math . 8 ] & \\ min \frac{1}{2} {ɛ}^{T} {Q}_{1} ɛ + \frac{1}{2} {ξ}^{T} {Q}_{2} ξ & ( 9 ) \\ s . t . U ξ \geq v & \end{matrix}$$

Here, ε is a difference between both sides of the upper part of the expression (8), and represents an equation error of the expression (8). ξ is a connected vector of fₑ and Δfᵥ and represents a variable vector. Q₁ and Q₂ are positive definite symmetric matrices that represent weights at minimization. Furthermore, inequality constraint of the expression (9) is used to express the constraint condition regarding the external force such as the vertical reaction force, friction cone, maximum value of the external force, or support polygon. For example, the inequality constraint regarding a rectangular support polygon is expressed by the following expression (10).

[Math. 9]

|Fₓ|≥μₜF_{z},

|F_{y}|≥μₜF_{z},

Fₓ≤0,

|Mₓ|≥d_{y}F_{z},

|M_{y}|≥dₓF_{z},

|M_{z}|≥μᵣF_{z}, (10)

Here, z represents a normal direction of a contact surface, and x and y represent orthogonal two-tangent directions perpendicular to z. (Fₓ, F_{y}, F_{z}) and (Mₓ, M_{y}, M₇) represent an external force and an external force moment acting on a contact point. μₜ and μᵣ are friction coefficients regarding translation and rotation, respectively. (dₓ, d_{y}) represents the size of the support polygon.

From the above expressions (9) and (10), solutions fₑ and Δfᵥ of a minimum norm or a minimum error are obtained. By substituting fₑ and Δfᵥ obtained from the above expression (9) into the lower part of the above expression (8), the joint force τₐ necessary for realizing the motion purpose can be obtained.

In a case of a system where a base is fixed and there is no non-drive joint, all virtual forces can be replaced only with the joint force, and fₑ=0 and Δfᵥ=0 can be set in the above expression (8). In this case, the following expression (11) can be obtained for the joint force τₐ from the lower part of the above expression (8).

[Math. 10]

τₐ=Jᵥₐ^{T}fᵥ (11)

The whole body coordination control using the generalized inverse dynamics according to the present embodiment has been described. By sequentially performing the virtual force calculation processing and the real force calculation processing as described above, the joint force τₐ for achieving a desired motion purpose can be obtained. In other words, conversely speaking, by reflecting the calculated joint force τₐ in a theoretical model in the motion of the joint units 421a to 421f, the joint units 421a to 421f are driven to achieve the desired motion purpose.

Note that, regarding the whole body coordination control using the generalized inverse dynamics described so far, in particular, details of the process of deriving the virtual force fᵥ, the method of solving the LCP to obtain the virtual force fᵥ, the solution of the QP problem, and the like, reference can be made to Japanese Patent Application Laid-Open Nos. 2009-95959 and 2010-188471, which are prior patent applications filed by the present applicant, for example.

<2-3. Ideal Joint Control>

Next, the ideal joint control according to the present embodiment will be described. The motion of each of the joint units 421a to 421f is modeled by the equation of motion of the second-order lag system of the following expression (12).

[Math. 11]

Iₐ\ddot{q}=τₐ+τₑ−vₐ\dot{q} (12)

Here, Iₐ represents moment of inertia (inertia) at the joint unit, τₐ represents the generated torque of the joint units 421a to 421f, τₑ represents external torque acting on each of the joint units 421a to 421f from the outside, and v_{c} represents a viscous drag coefficient in each of the joint units 421a to 421f. The above expression (12) can also be said to be a theoretical model that represents the motion of the actuators in the joint units 421a to 421f.

τₐ that is the real force to act on each of the joint units 421a to 421f for realizing the motion purpose can be calculated using the motion purpose and the constraint condition by the operation using the generalized inverse dynamics described in <2-2. Generalized Inverse Dynamics> above. Therefore, ideally, by applying each calculated τₐ to the above expression (12), a response according to the theoretical model illustrated in the above expression (12) is realized, in other words, the desired motion purpose should be achieved.

However, in practice, errors (modeling errors) may occur between the motions of the joint units 421a to 421f and the theoretical model as illustrated in the above expression (12), due to the influence of various types of disturbance. The modeling errors can be roughly classified into those due to mass property such as weight, center of gravity, inertia tensor of the multilink structure, and those due to friction, inertia, and the like inside joint units 421a to 421f Among them, the modeling errors due to the former mass property can be relatively easily reduced at the time of constructing the theoretical model by improving the accuracy of computer aided design (CAD) data and applying an identification method.

Meanwhile, the modeling errors due to the latter friction, inertia, and the like inside the joint units 421a to 421f are caused by phenomena that are difficult to model, such as friction in a reduction gear 426 of the joint units 421a to 421f, for example, and a modeling error that cannot be ignored may remain during model construction. Furthermore, there is a possibility that an error occurs between the values of the inertia Iₐ and the viscous drag coefficient vₑ in the above expression (12) and the values in the actual joint units 421a to 421f. These errors that are difficult to model can become the disturbance in the drive control of the joint units 421a to 421f. Therefore, in practice, the motions of the joint units 421a to 421f may not respond according to the theoretical model illustrated in the above expression (12), due to the influence of such disturbance. Therefore, even when the real force τₐ, which is a joint force calculated by the generalized inverse dynamics, is applied, there may be a case where the motion purpose that is the control target is not achieved. In the present embodiment, correcting the responses of the joint units 421a to 421f so as to perform ideal responses according to the theoretical model illustrated in the above expression (12), by adding an active control system to each of the joint units 421a to 421f, is considered. Specifically, in the present embodiment, not only performing friction compensation type torque control using the torque sensors 428 and 428a of the joint units 421a to 421f but also performing an ideal response according to the theoretical values up to the inertia Iₐ and the viscous drag coefficient vₐ to the required generated torque TI and external torque τₑ.

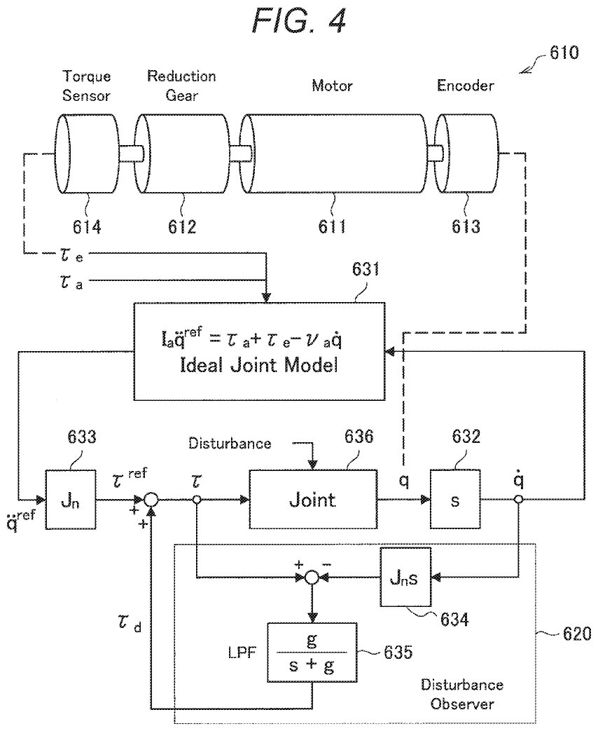

In the present embodiment, control of the driving of the joint units 421a to 421f of the support arm device 400 to perform ideal responses as described in the above expression (12) is called ideal joint control. Here, in the following description, an actuator controlled to be driven by the ideal joint control is also referred to as a virtualized actuator (VA) because of performing an ideal response. Hereinafter, the ideal joint control according to the present embodiment will be described with reference to FIG. 4.

FIG. 4 is an explanatory diagram for describing the ideal joint control according to an embodiment of the present disclosure. Note that FIG. 4 schematically illustrates a conceptual arithmetic unit that performs various operations regarding the ideal joint control in blocks.

Here, a response of an actuator 610 according to the theoretical model expressed by the above expression (12) is nothing less than achievement of the rotation angular acceleration on the left side when the right side of the expression (12) is given. Furthermore, as illustrated in the above expression (12), the theoretical model includes an external torque term z acting on the actuator 610. In the present embodiment, the external torque term e is measured by a torque sensor 614 in order to perform the ideal joint control. Furthermore, a disturbance observer 620 is applied to calculate a disturbance estimation value d that is an estimation value of a torque due to disturbance on the basis of a rotation angle q of the actuator 610 measured by an encoder 613.

A block 631 represents an arithmetic unit that performs an operation according to an ideal joint model of the joint units 421a to 421f illustrated in the above expression (12). The block 631 can output a rotation angular acceleration target value (a second derivative of a rotation angle target value q^{ref} described on the left side of the above expression (12), using the generated torque τₐ, the external torque τₑ, and the rotation angular speed (first-order differentiation of the rotation angle q) as inputs.

In the present embodiment, the generated torque τₐ calculated by the method described in <2-2. Generalized inverse dynamics> above and the external torque τₑ measured by the torque sensor 614 are input to the block 631. Meanwhile, when the rotation angle q measured by the encoder 613 is input to a block 632 representing an arithmetic unit that performs a differential operation, the rotation angular speed (the first-order differentiation of the rotation angle q) is calculated. When the rotation angular speed calculated in the block 632 is input to the block 631 in addition to the generated torque τₐ and the external torque τₑ, the rotation angular acceleration target value is calculated by the block 631. The calculated rotation angular acceleration target value is input to a block 633.

The block 633 represents an arithmetic unit that calculates a torque generated in the actuator 610 on the basis of the rotation angular acceleration of the actuator 610. In the present embodiment, specifically, the block 633 can obtain a torque target value τ^{ref} by multiplying the rotation angular acceleration target value by nominal inertia Jₙ in the actuator 610. In the ideal response, the desired motion purpose should be achieved by causing the actuator 610 to generate the torque target value τ^{ref}. However, as described above, there is a case where the influence of the disturbance or the like occurs in the actual response. Therefore, in the present embodiment, the disturbance observer 620 calculates the disturbance estimation value τ_{d} and corrects the torque target value τ^{ref} using the disturbance estimation value τ_{d}.

A configuration of the disturbance observer 620 will be described. As illustrated in FIG. 4, the disturbance observer 620 calculates the disturbance estimation value τ_{d} On the basis of the torque command value τ and the rotation angular speed output from the rotation angle q measured by the encoder 613. Here, the torque command value τ is a torque value to be finally generated in the actuator 610 after the influence of disturbance is corrected. For example, win a case where the disturbance estimation value τ_{d} is not calculated, the torque command value τ becomes the torque target value τ^{ref}.

The disturbance observer 620 includes a block 634 and a block 635. The block 634 represents an arithmetic unit that calculates a torque generated in the actuator 610 on the basis of the rotation angular speed of the actuator 610. In the present embodiment, specifically, the rotation angular speed calculated by the block 632 from the rotation angle q measured by the encoder 613 is input to the block 634. The block 634 obtains the rotation angular acceleration by performing an operation represented by a transfer function Jₙs, in other words, by differentiating the rotation angular speed, and further multiplies the calculated rotation angular acceleration by the nominal inertia Jₙ, thereby calculating an estimation value of the torque actually acting on the actuator 610 (torque estimation value).

In the disturbance observer 620, the difference between the torque estimation value and the torque command value τ is obtained, whereby the disturbance estimation value τ_{d}, which is the value of the torque due to the disturbance, is estimated. Specifically, the disturbance estimation value τ_{d} may be a difference between the torque command value τ in the control of the preceding cycle and the torque estimation value in the current control. Since the torque estimation value calculated by the block 634 is based on the actual measurement value and the torque command value τ calculated by the block 633 is based on the ideal theoretical model of the joint units 421a to 421f illustrated in the block 631, the influence of the disturbance, which is not considered in the theoretical model, can be estimated by taking the difference between the torque estimation value and the torque command value τ.

Furthermore, the disturbance observer 620 is provided with a low pass filter (LPF) illustrated in a block 635 to prevent system divergence. The block 635 outputs only a low frequency component to the input value by performing an operation represented by a transfer function g/(s+g) to stabilize the system. In the present embodiment, the difference value between the torque estimation value and the torque command value τ^{ref} calculated by the block 634 is input to the block 635, and a low frequency component of the difference value is calculated as the disturbance estimation value τ_{d}.

In the present embodiment, feedforward control to add the disturbance estimation value τ_{d} calculated by the disturbance observer 620 to the torque target value τ^{ref} is performed, whereby the torque command value τ that is the torque value to be finally generated in the actuator 610 is calculated. Then, the actuator 610 is driven on the basis of the torque command value r. Specifically, the torque command value τ is converted into a corresponding current value (current command value), and the current command value is applied to a motor 611, so that the actuator 610 is driven.

As described above, with the configuration described with reference to FIG. 4, the response of the actuator 610 can be made to follow the target value even in a case where there is a disturbance component such as friction in the drive control of the joint units 421a to 421f according to the present embodiment. Furthermore, with regard to the drive control of the joint units 421a to 421 f, an ideal response according to the inertia Iₐ and the viscous drag coefficient vₐ assumed by the theoretical model can be made.

Note that, for details of the above-described ideal joint control, Japanese Patent Application Laid-Open No. 2009-269102, which is a prior patent application filed by the present applicant, can be referred to, for example.

The generalized inverse dynamics used in the present embodiment has been described, and the ideal joint control according to the present embodiment has been described with reference to FIG. 4. As described above, in the present embodiment, the whole body coordination control, in which the drive parameters of the joint units 421a to 421f (for example, the generated torque values of the joint units 421a to 421f) for achieving the motion purpose of the arm unit 420 are calculated in consideration of the constraint condition, is performed using the generalized inverse dynamics. Furthermore, as described with reference to FIG. 4, in the present embodiment, the ideal joint control that realizes the ideal response based on the theoretical model in the drive control of the joint units 421a to 421f by performing correction of the generated torque value, which has been calculated in the whole body coordination control using the generalized inverse dynamics, in consideration of the influence of the disturbance, is performed. Therefore, in the present embodiment, highly accurate drive control that achieves the motion purpose becomes possible with regard to the driving of the arm unit 420.

<2-4. Configuration of Robot Arm Control System>

Next, a configuration of a robot arm control system according to the present embodiment, in which the whole body coordination control and the ideal joint control described in <2-2. Generalized Inverse Dynamics> and <2-3. Ideal Joint Control> above are applied to drive control of a robot arm device will be described.

A configuration example of a robot arm control system according to an embodiment of the present disclosure will be described with reference to FIG. 5. FIG. 5 is a functional block diagram illustrating a configuration example of a robot arm control system according to an embodiment of the present disclosure. Note that, in the robot arm control system illustrated in FIG. 5, a configuration related to drive control of an arm unit of a robot arm device will be mainly illustrated.

Referring to FIG. 5, a robot arm control system 1 according to an embodiment of the present disclosure includes a robot arm device 10, a control device 20, and a display device 30. In the present embodiment, the control device 20 performs various operations in the whole body coordination control described in <2-2. Generalized Inverse Dynamics> and the ideal joint control described in <2-3. Ideal Joint Control> above, and driving of the arm unit of the robot arm device 10 is controlled on the basis of an operation result. Furthermore, the arm unit of the robot arm device 10 is provided with an imaging unit 140 described below, and an image captured by the imaging unit 140 is displayed on a display screen of the display device 30. Hereinafter, configurations of the robot arm device 10, the control device 20, and the display device 30 will be described in detail.

The robot arm device 10 includes the arm unit that is a multilink structure including a plurality of joint units and a plurality of links, and drives the arm unit within a movable range to control the position and posture of a distal end unit provided at a distal end of the arm unit. The robot arm device 10 corresponds to the support arm device 400 illustrated in FIG. 3.

Referring to FIG. 5, the robot arm device 10 includes an arm control unit 110 and an arm unit 120. Furthermore, the arm unit 120 includes a joint unit 130 and the imaging unit 140.

The arm control unit 110 integrally controls the robot arm device 10 and controls driving of the arm unit 120. The arm control unit 110 corresponds to the control unit (not illustrated in FIG. 3) described with reference to FIG. 3. Specifically, the arm control unit 110 includes a drive control unit 111. Driving of the joint unit 130 is controlled by the control of the drive control unit 111, so that the driving of the arm unit 120 is controlled. More specifically, the drive control unit 111 controls a current amount to be supplied to a motor in an actuator of the joint unit 130 to control the number of rotations of the motor, thereby controlling a rotation angle and generated torque in the joint unit 130. However, as described above, the drive control of the arm unit 120 by the drive control unit 111 is performed on the basis of the operation result in the control device 20. Therefore, the current amount to be supplied to the motor in the actuator of the joint unit 130, which is controlled by the drive control unit 111, is a current amount determined on the basis of the operation result in the control device 20.

The arm unit 120 is a multilink structure including a plurality of joints and a plurality of links, and driving of the arm unit 120 is controlled by the control of the arm control unit 110. The arm unit 120 corresponds to the arm unit 420 illustrated in FIG. 3. The arm unit 120 includes the joint unit 130 and the imaging unit 140. Note that, since functions and structures of the plurality of joint units included in the arm unit 120 are similar to one another, FIG. 5 illustrates a configuration of one joint unit 130 as a representative of the plurality of joint units.

The joint unit 130 rotatably connects the links with each other in the arm unit 120, and drives the arm unit 120 as rotational driving of the joint unit 130 is controlled by the control of the arm control unit 110. The joint unit 130 corresponds to the joint units 421a to 421f illustrated in FIG. 3. Furthermore, the joint unit 130 includes an actuator.

The joint unit 130 includes a joint drive unit 131 and a joint state detection unit 132.

The joint drive unit 131 is a drive mechanism in the actuator of the joint unit 130, and the joint unit 130 is rotationally driven as the joint drive unit 131 is driven. The driving of the joint drive unit 131 is controlled by the drive control unit 111. For example, the joint drive unit 131 is a configuration corresponding to the motor and a motor driver, and the joint drive unit 131 being driven corresponds to the motor driver driving the motor with the current amount according to a command from the drive control unit 111.

The joint state detection unit 132 detects a state of the joint unit 130. Here, the state of the joint unit 130 may mean a state of motion of the joint unit 130. For example, the state of the joint unit 130 includes information of the rotation angle, rotation angular speed, rotation angular acceleration, generated torque of the joint unit 130, and the like. In the present embodiment, the joint state detection unit 132 has a rotation angle detection unit 133 that detects the rotation angle of the joint unit 130 and a torque detection unit 134 that detects the generated torque and the external torque of the joint unit 130. Note that the rotation angle detection unit 133 and the torque detection unit 134 correspond to an encoder and a torque sensor of the actuator, respectively. The joint state detection unit 132 transmits the detected state of the joint unit 130 to the control device 20.

The imaging unit 140 is an example of the distal end unit provided at the distal end of the arm unit 120, and acquires an image of a capture target. The imaging unit 140 corresponds to the imaging unit 423 illustrated in FIG. 3. Specifically, the imaging unit 140 is a camera or the like that can capture the capture target in the form of a moving image or a still image. More specifically, the imaging unit 140 includes a plurality of light receiving elements arranged in a two dimensional manner, and can obtain an image signal representing an image of the capture target by photoelectric conversion in the light receiving elements. The imaging unit 140 transmits the acquired image signal to the display device 30.

Note that, as in the case of the support arm device 400 illustrated in FIG. 3, the imaging unit 423 is provided at the distal end of the arm unit 420, the imaging unit 140 is actually provided at the distal end of the arm unit 120 in the robot arm device 10. FIG. 5 illustrates a state in which the imaging unit 140 is provided at a distal end of a final link via the plurality of joint units 130 and the plurality of links by schematically illustrating a link between the joint unit 130 and the imaging unit 140.

Note that, in the present embodiment, various medical instruments can be connected to the distal end of the arm unit 120 as the distal end unit. Examples of the medical instruments include various treatment instruments such as a scalpel and forceps, and various units used in treatment, such as a unit of various detection devices such as probes of an ultrasonic examination device. Furthermore, in the present embodiment, the imaging unit 140 illustrated in FIG. 5 or a unit having an imaging function such as an endoscope or a microscope may also be included in the medical instruments. Thus, the robot arm device 10 according to the present embodiment can be said to be a medical robot arm device provided with medical instruments. Similarly, the robot arm control system 1 according to the present embodiment can be said to be a medical robot arm control system. Note that the robot arm device 10 illustrated in FIG. 5 can also be said to be a VM robot arm device provided with a unit having an imaging function as the distal end unit. Furthermore, a stereo camera having two imaging units (camera units) may be provided at the distal end of the arm unit 120, and may capture an imaging target to be displayed as a 3D image.

The function and configuration of the robot arm device 10 have been described above. Next, a function and a configuration of the control device 20 will be described. Referring to FIG. 5, the control device 20 includes an input unit 210, a storage unit 220, and a control unit 230.

The control unit 230 integrally controls the control device 20 and performs various operations for controlling the driving of the arm unit 120 in the robot arm device 10. Specifically, to control the driving of the arm unit 120 of the robot arm device 10, the control unit 230 performs various operations in the whole body coordination control and the ideal joint control. Hereinafter, the function and configuration of the control unit 230 will be described in detail. The whole body coordination control and the ideal joint control have been already described in <2-2. Generalized Inverse Dynamics> and <2-3. Ideal Joint Control> above, and thus detailed description is omitted here.

The control unit 230 includes a whole body coordination control unit 240 and an ideal joint control unit 250.

The whole body coordination control unit 240 performs various operations regarding the whole body coordination control using the generalized inverse dynamics. In the present embodiment, the whole body coordination control unit 240 acquires a state (arm state) of the arm unit 120 on the basis of the state of the joint unit 130 detected by the joint state detection unit 132. Furthermore, the whole body coordination control unit 240 calculates a control value for the whole body coordination control of the arm unit 120 in an operation space, using the generalized inverse dynamics, on the basis of the arm state, and a motion purpose and a constraint condition of the arm unit 120. Note that the operation space is a space for describing the relationship between the force acting on the arm unit 120 and the acceleration generated in the arm unit 120, for example.

The whole body coordination control unit 240 includes an arm state acquisition unit 241, an arithmetic condition setting unit 242, a virtual force calculation unit 243, and a real force calculation unit 244.

The arm state acquisition unit 241 acquires the state (arm state) of the arm unit 120 on the basis of the state of the joint unit 130 detected by the joint state detection unit 132. Here, the arm state may mean the state of motion of the arm unit 120. For example, the arm state includes information such as the position, speed, acceleration, and force of the arm unit 120. As described above, the joint state detection unit 132 acquires, as the state of the joint unit 130, the information of the rotation angle, rotation angular speed, rotation angular acceleration, generated torque in each joint unit 130, and the like. Furthermore, although to be described below, the storage unit 220 stores various types of information to be processed by the control device 20. In the present embodiment, the storage unit 220 may store various types of information (arm information) regarding the arm unit 120, for example, the number of joint units 130 and links configuring the arm unit 120, connection states between the links and the joint units 130, and lengths of the links, and the like. The arm state acquisition unit 241 can acquire the arm information from the storage unit 220. Therefore, the arm state acquisition unit 241 can acquire, as the arm state, information such as the positions (coordinates) in the space of the plurality of joint units 130, the plurality of links, and the imaging unit 140 (in other words, the shape of the arm unit 120 and the position and posture of the imaging unit 140), and the forces acting on the joint units 130, the links, and the imaging unit 140, on the basis of the state and the arm information of the joint units 130. The arm state acquisition unit 241 transmits the acquired arm information to the arithmetic condition setting unit 242.

The arithmetic condition setting unit 242 sets operation conditions in an operation regarding the whole body coordination control using the generalized inverse dynamics. Here, the operation condition may be a the motion purpose and a constraint condition. The motion purpose may be various types of information regarding the motion of the arm unit 120. Specifically, the motion purpose may be target values of the position and posture (coordinates), speed, acceleration, force, and the like of the imaging unit 140, or target values of the positions and postures (coordinates), speeds, accelerations, forces, and the like of the plurality of joint units 130 and the plurality of links of the arm unit 120. Furthermore, the constraint condition may be various types of information that restricts (restrains) the motion of the arm unit 120. Specifically, the constraint condition may be coordinates of a region where each configuration component of the arm unit cannot move, an unmovable speed, a value of acceleration, a value of an ungenerable force, and the like. Furthermore, restriction ranges of various physical quantities under the constraint condition may be set according to inability to structurally realizing the arm unit 120 or may be appropriately set by the user. Furthermore, the arithmetic condition setting unit 242 includes a physical model for the structure of the arm unit 120 (in which, for example, the number and lengths of the links configuring the arm unit 120, the connection states of the links via the joint units 130, the movable ranges of the joint units 130, and the like are modeled), and may set a motion condition and the constraint condition by generating a control model in which the desired motion condition and constraint condition are reflected in the physical model.

In the present embodiment, appropriate setting of the motion purpose and the constraint condition enables the arm unit 120 to perform a desired operation. For example, not only can the imaging unit 140 be moved to a target position by setting a target value of the position of the imaging unit 140 as the motion purpose but also the arm unit 120 can be driven by providing a constraint of movement by the constraint condition to prevent the arm unit 120 from intruding into a predetermined region in the space.

A specific example of the motion purpose includes, for example, a pivot operation, which is a turning operation with an axis of a cone serving as a pivot axis, in which the imaging unit 140 moves in a conical surface setting an operation site as a top in a state where the capture direction of the imaging unit 140 is fixed to the operation site. Furthermore, in the pivot operation, the turning operation may be performed in a state where the distance between the imaging unit 140 and a point corresponding to the top of the cone is kept constant. By performing such a pivot operation, an observation site can be observed from an equal distance and at different angles, whereby the convenience of the user who performs surgery can be improved.

Furthermore, as another specific example, the motion purpose may be content to control the generated torque in each joint unit 130. Specifically, the motion purpose may be a power assist operation to control the state of the joint unit 130 to cancel the gravity acting on the arm unit 120, and further control the state of the joint unit 130 to support the movement of the arm unit 120 in a direction of a force provided from the outside. More specifically, in the power assist operation, the driving of each joint unit 130 is controlled to cause each joint unit 130 to generate a generated torque that cancels the external torque due to the gravity in each joint unit 130 of the arm unit 120, whereby the position and posture of the arm unit 120 are held in a predetermined state. In a case where an external torque is further added from the outside (for example, from the user) in the aforementioned state, the driving of each joint unit 130 is controlled to cause each joint unit 130 to generate a generated torque in the same direction as the added external torque. By performing such a power assist operation, the user can move the arm unit 120 with a smaller force in a case where the user manually moves the arm unit 120. Therefore, a feeling as if the user moved the arm unit 120 under weightlessness can be provided to the user. Furthermore, the above-described pivot operation and the power assist operation can be combined.

Here, in the present embodiment, the motion purpose may mean an operation (motion) of the arm unit 120 realized by the whole body coordination control or may mean an instantaneous motion purpose in the operation (in other words, a target value in the motion purpose). For example, in the above-described pivot operation, the imaging unit 140 performing the pivot operation itself is the motion purpose. In the act of performing the pivot operation, values of the position, speed, and the like of the imaging unit 140 in a conical surface in the pivot operation are set as the instantaneous motion purpose (the target values in the motion purpose). Furthermore, in the above-described power assist operation, for example, performing the power assist operation to support the movement of the arm unit 120 in the direction of the force applied from the outside itself is the motion purpose. In the act of performing the power assist operation, the value of the generated torque in the same direction as the external torque applied to each joint unit 130 is set as the instantaneous motion purpose (the target value in the motion purpose). The motion purpose in the present embodiment is a concept including both the instantaneous motion purpose (for example, the target values of the positions, speeds, forces, and the like of the configuration members of the arm unit 120 at a certain time) and the operations of the configuration members of the arm unit 120 realized over time as a result of the instantaneous motion purpose having been continuously achieved. The instantaneous motion purpose is set each time in each step in an operation for the whole body coordination control in the whole body coordination control unit 240, and the operation is repeatedly performed, so that the desired motion purpose is finally achieved.

Note that, in the present embodiment, the viscous drag coefficient in a rotation motion of each joint unit 130 may be appropriately set when the motion purpose is set. As described above, the joint unit 130 according to the present embodiment is configured to be able to appropriately adjust the viscous drag coefficient in the rotation motion of the actuator. Therefore, by setting the viscous drag coefficient in the rotation motion of each joint unit 130 when setting the motion purpose, an easily rotatable state or a less easily rotatable state can be realized for the force applied from the outside, for example. For example, in the above-descried power assist operation, when the viscous drag coefficient in the joint unit 130 is set to be small, a force required by the user to move the arm unit 120 can be made small, and a weightless feeling provided to the user can be promoted. As described above, the viscous drag coefficient in the rotation motion of each joint unit 130 may be appropriately set according to the content of the motion purpose.

Here, in the present embodiment, as will be described below, the storage unit 220 may store parameters regarding the operation conditions such as the motion purpose and the constraint condition used in the operation regarding the whole body coordination control. The arithmetic condition setting unit 242 can set the constraint condition stored in the storage unit 220 as the constraint condition used for the operation of the whole body coordination control.

Furthermore, in the present embodiment, the arithmetic condition setting unit 242 can set the motion purpose by a plurality of methods. For example, the arithmetic condition setting unit 242 may set the motion purpose on the basis of the arm state transmitted from the arm state acquisition unit 241. As described above, the arm state includes information of the position of the arm unit 120 and information of the force acting on the arm unit 120. Therefore, for example, in a case where the user is trying to manually move the arm unit 120, information regarding how the user is moving the arm unit 120 is also acquired by the arm state acquisition unit 241 as the arm state. Therefore, the arithmetic condition setting unit 242 can set the position, speed, force, and the like to/at/with which the user has moved the arm unit 120, as the instantaneous motion purpose, on the basis of the acquired arm state. By thus setting the motion purpose, the driving of the arm unit 120 is controlled to follow and support the movement of the arm unit 120 by the user.

Furthermore, for example, the arithmetic condition setting unit 242 may set the motion purpose on the basis of an instruction input from the input unit 210 by the user. Although to be described below, the input unit 210 is an input interface for the user to input information, commands, and the like regarding the drive control of the robot arm device 10, to the control device 20. In the present embodiment, the motion purpose may be set on the basis of an operation input from the input unit 210 by the user. Specifically, the input unit 210 has, for example, operation means operated by the user, such as a lever and a pedal. The positions, speeds, and the like of the configuration members of the arm unit 120 may be set as the instantaneous motion purpose by the arithmetic condition setting unit 242 in response to an operation of the lever, pedal, or the like.

Moreover, for example, the arithmetic condition setting unit 242 may set the motion purpose stored in the storage unit 220 as the motion purpose used for the operation of the whole body coordination control. For example, in the case of the motion purpose that the imaging unit 140 stands still at a predetermined point in the space, coordinates of the predetermined point can be set in advance as the motion purpose. Furthermore, for example, in the case of the motion purpose that the imaging unit 140 moves on a predetermined trajectory in the space, coordinates of each point representing the predetermined trajectory can be set in advance as the motion purpose. As described above, in a case where the motion purpose can be set in advance, the motion purpose may be stored in the storage unit 220 in advance. Furthermore, in the case of the above-described pivot operation, for example, the motion purpose is limited to a motion purpose setting the position, speed, and the like in the conical surface as the target values. In the case of the power assist operation, the motion purpose is limited to a motion purpose setting the force as the target value. In the case where the motion purpose such as the pivot operation or the power assist operation is set in advance in this way, information regarding ranges, types and the like of the target values settable as the instantaneous motion purpose in such a motion purpose may be stored in the storage unit 220. The arithmetic condition setting unit 242 can also set the various types of information regarding such a motion purpose as the motion purpose.

Note that by which method the arithmetic condition setting unit 242 sets the motion purpose may be able to be appropriately set by the user according to the application of the robot arm device 10 or the like. Furthermore, the arithmetic condition setting unit 242 may set the motion purpose and the constraint condition by appropriately combining the above-described methods. Note that a priority of the motion purpose may be set in the constraint condition stored in the storage unit 220, or in a case where is a plurality of motion purposes different from one another, the arithmetic condition setting unit 242 may set the motion purpose according to the priority of the constraint condition. The arithmetic condition setting unit 242 transmits the arm state and the set motion purpose and constraint condition to the virtual force calculation unit 243.

The virtual force calculation unit 243 calculates a virtual force in the operation regarding the whole body coordination control using the generalized inverse dynamics. The processing of calculating the virtual force performed by the virtual force calculation unit 243 may be the series of processing described in, for example, <2-2-1. Virtual Force Calculation Processing> above. The virtual force calculation unit 243 transmits the calculated virtual force fᵥ to the real force calculation unit 244.

The real force calculation unit 244 calculates a real force in the operation regarding the whole body coordination control using the generalized inverse dynamics. The processing of calculating the real force performed by the real force calculation unit 244 may be the series of processing described in, for example, <2-2-2. Real Force Calculation Processing> above. The real force calculation unit 244 transmits the calculated real force (generated torque) τₐ to the ideal joint control unit 250. Note that, in the present embodiment, the generated torque τₐ calculated by the real force calculation unit 244 is also referred to as a control value or a control torque value in the sense of a control value of the joint unit 130 in the whole body coordination control.

The ideal joint control unit 250 performs various operations regarding the ideal joint control using the generalized inverse dynamics. In the present embodiment, the ideal joint control unit 250 correct the influence of disturbance for the generated torque τₐ calculated by the real force calculation unit 244 to calculate a torque command value τ realizing an ideal response of the arm unit 120. Note that the arithmetic processing performed by the ideal joint control unit 250 corresponds to the series of processing described in <2-3. Ideal Joint Control> above.

The ideal joint control unit 250 includes a disturbance estimation unit 251 and a command value calculation unit 252.

The disturbance estimation unit 251 calculates a disturbance estimation value τ_{d} on the basis of the torque command value τ and the rotation angular speed calculated from the rotation angle q detected by the rotation angle detection unit 133. Note that the torque command value τ mentioned here is a command value that represents the generated torque in the arm unit 120 to be finally transmitted to the robot arm device 10. Thus, the disturbance estimation unit 251 has a function corresponding to the disturbance observer 620 illustrated in FIG. 4.

The command value calculation unit 252 calculates the torque command value τ that is a command value representing the torque to be generated in the arm unit 120 and finally transmitted to the robot arm device 10, using the disturbance estimation value τ_{d} calculated by the disturbance estimation unit 251. Specifically, the command value calculation unit 252 adds the disturbance estimation value τ_{d} calculated by the disturbance estimation unit 251 to τ^{ref} calculated from the ideal model of the joint unit 130 described in the above expression (12) to calculate the torque command value τ. For example, win a case where the disturbance estimation value τ_{d} is not calculated, the torque command value τ becomes the torque target value τ^{ref}. Thus, the function of the command value calculation unit 252 corresponds to the function other than the disturbance observer 620 illustrated in FIG. 4.

As described above, in the ideal joint control unit 250, the information is repeatedly exchanged between the disturbance estimation unit 251 and the command value calculation unit 252, so that the series of processing described with reference to FIG. 4 is performed. The ideal joint control unit 250 transmits the calculated torque command value τ to the drive control unit 111 of the robot arm device 10. The drive control unit 111 performs control to supply the current amount corresponding to the transmitted torque command value τ to the motor in the actuator of the joint unit 130, thereby controlling the number of rotations of the motor and controlling the rotation angle and the generated torque in the joint unit 130.

In the robot arm control system 1 according to the present embodiment, the drive control of the arm unit 120 in the robot arm device 10 is continuously performed during work using the arm unit 120, so the above-described processing in the robot arm device 10 and the control device 20 is repeatedly performed. In other words, the state of the joint unit 130 is detected by the joint state detection unit 132 of the robot arm device 10 and transmitted to the control device 20. The control device 20 performs various operations regarding the whole body coordination control and the ideal joint control for controlling the driving of the arm unit 120 on the basis of the state of the joint unit 130, and the motion purpose and the constraint condition, and transmits the torque command value T as the operation result to the robot arm device 10. The robot arm device 10 controls the driving of the arm unit 120 on the basis of the torque command value τ, and the state of the joint unit 130 during or after the driving is detected by the joint state detection unit 132 again.

Description about other configurations included in the control device 20 will be continued.

The input unit 210 is an input interface for the user to input information, commands, and the like regarding the drive control of the robot arm device 10 to the control device 20. In the present embodiment, the driving of the arm unit 120 of the robot arm device 10 may be controlled on the basis of the operation input from the input unit 210 by the user, and the position and posture of the imaging unit 140 may be controlled. Specifically, as described above, instruction information regarding the instruction of the driving of the arm input from the input unit 210 by the user is input to the arithmetic condition setting unit 242, so that the arithmetic condition setting unit 242 may set the motion purpose in the whole body coordination control on the basis of the instruction information. The whole body coordination control is performed using the motion purpose based on the instruction information input by the user as described above, so that the driving of the arm unit 120 according to the operation input of the user is realized.

Specifically, the input unit 210 includes operation means operated by the user, such as a mouse, a keyboard, a touch panel, a button, a switch, a lever, and a pedal, for example. For example, in a case where the input unit 210 has a pedal, the user can control the driving of the arm unit 120 by operating the pedal with the foot. Therefore, even in a case where the user is performing treatment using both hands on the operation site of the patient, the user can adjust the position and posture of the imaging unit 140, in other words, the user can adjust a capture position and a capture angle of the operation site, by the operation of the pedal with the foot.

The storage unit 220 stores various types of information processed by the control device 20. In the present embodiment, the storage unit 220 can store various parameters used in the operation regarding the whole body coordination control and the ideal joint control performed by the control unit 230. For example, the storage unit 220 may store the motion purpose and the constraint condition used in the operation regarding the whole body coordination control by the whole body coordination control unit 240. The motion purpose stored in the storage unit 220 may be, as described above, a motion purpose that can be set in advance, such as, for example, the imaging unit 140 standing still at a predetermined point in the space. Furthermore, the constraint conditions may be set in advance by the user and stored in the storage unit 220 according to a geometric configuration of the arm unit 120, the application of the robot arm device 10, and the like. Furthermore, the storage unit 220 may also store various types of information regarding the arm unit 120 used when the arm state acquisition unit 241 acquires the arm state. Moreover, the storage unit 220 may store the operation result, various numerical values, and the like calculated in the operation process in the operation regarding the whole body coordination control and the ideal joint control by the control unit 230. As described above, the storage unit 220 may store any parameters regarding the various types of processing performed by the control unit 230, and the control unit 230 can performs various types of processing while mutually exchanging information with the storage unit 220.

The function and configuration of the control device 20 have been described above. Note that the control device 20 according to the present embodiment can be configured by, for example, various information processing devices (arithmetic processing devices) such as a personal computer (PC) and a server. Next, a function and a configuration of the display device 30 will be described.

The display device 30 displays the information on the display screen in various formats such as texts and images to visually notify the user of various types of information. In the present embodiment, the display device 30 displays the image captured by the imaging unit 140 of the robot arm device 10 on the display screen. Specifically, the display device 30 has functions and configurations of an image signal processing unit (not illustrated) that applies various types of image processing to an image signal acquired by the imaging unit 140, a display control unit (not illustrated) that performs control to display an image based on the processed image signal on the display screen, and the like. Note that the display device 30 may have various functions and configurations that a display device generally has, in addition to the above-described functions and configurations. The display device 30 corresponds to the display device 5041 illustrated in FIG. 1.

The functions and configurations of the robot arm device 10, the control device 20, and the display device 30 according to the present embodiment have been described above with reference to FIG. 5. Each of the above-described constituent elements may be configured using general-purpose members or circuit, or may be configured by hardware specialized for the function of each constituent element. Furthermore, all the functions of the configuration elements may be performed by a CPU or the like. Therefore, the configuration to be used can be changed as appropriate according to the technical level of the time of carrying out the present embodiment.

As described above, according to the present embodiment, the arm unit 120 that is the multilink structure in the robot arm device 10 has at least six degrees or more of freedom, and the driving of each of the plurality of joint units 130 configuring the arm unit 120 is controlled by the drive control unit 111. Then, a medical instrument is provided at the distal end of the arm unit 120. The driving of each of the joint units 130 is controlled as described above, so that the drive control of the arm unit 120 with a higher degree of freedom is realized, and the medical robot arm device 10 with higher operability for the user is realized.

More specifically, according to the present embodiment, the joint state detection unit 132 detects the state of the joint unit 130 in the robot arm device 10. Then, the control device 20 performs various operations regarding the whole body coordination control using the generalized inverse dynamics for controlling the driving of the arm unit 120 on the basis of the state of the joint unit 130, and the motion purpose and the constraint condition, and calculates the torque command value τ as the operation result. Moreover, the robot arm device 10 controls the driving of the arm unit 120 on the basis of the torque command value τ. As described above, in the present embodiment, the driving of the arm unit 120 is controlled by the whole body coordination control using the generalized inverse dynamics. Therefore, the drive control of the arm unit 120 by force control is realized, and a robot arm device with higher operability for the user is realized. Furthermore, in the present embodiment, control to realize various motion purposes for further improving the convenience of the user, such as the pivot operation and the power assist operation, is possible in the whole body coordination control. Moreover, in the present embodiment, various driving means are realized, such as manually moving the arm unit 120, and moving the arm unit 120 by the operation input from a pedal. Therefore, further improvement of the convenience for the user is realized.

Furthermore, in the present embodiment, the ideal joint control is applied together with the whole body coordination control to the drive control of the arm unit 120. In the ideal joint control, the disturbance components such as friction and inertia inside the joint unit 130 are estimated, and the feedforward control using the estimated disturbance components is performed. Therefore, even in a case where there is a disturbance component such as friction, an ideal response can be realized for the driving of the joint unit 130. Therefore, in the drive control of the arm unit 120, highly accurate response and high positioning accuracy and stability with less influence of vibration and the like are realized.

Moreover, in the present embodiment, each of the plurality of joint units 130 configuring the arm unit 120 has a configuration adapted to the ideal joint control, and the rotation angle, generated torque and viscous drag coefficient in each joint unit 130 can be controlled with the current value. As described above, the driving of each joint unit 130 is controlled with the current value, and the driving of each joint unit 130 is controlled while grasping the state of the entire arm unit 120 by the whole body coordination control. Therefore, counterbalance is unnecessary and downsizing of the robot arm device 10 is realized.

<<3. Basic Configuration of Oblique-Viewing Endoscope>>

Next, a basic configuration of an oblique-viewing endoscope will be described as an example of the endoscope.

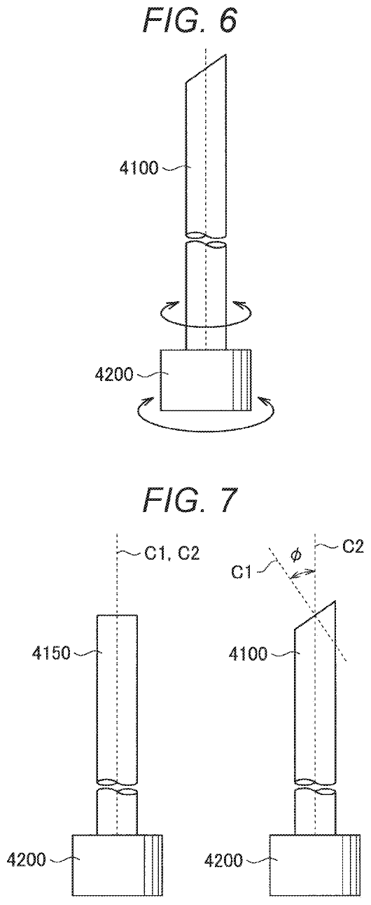

FIG. 6 is a schematic view illustrating a configuration of an oblique-viewing endoscope 4100 according to an embodiment of the present disclosure. As illustrated in FIG. 6, the oblique-viewing endoscope 4100 is attached to a distal end of a camera head 4200. The oblique-viewing endoscope 4100 corresponds to the lens barrel 5003 described in FIGS. 1 and 2, and the camera head 4200 corresponds to the camera head 5005 described in FIGS. 1 and 2. The oblique-viewing endoscope 4100 and the camera head 4200 are rotatable independently of each other. An actuator is provided between the oblique-viewing endoscope 4100 and the camera head 4200, similarly to the joint units 5033a. 5033b, and 5033c, and the oblique-viewing endoscope 4100 rotates with respect to the camera head 4200 by driving of the actuator. Thereby, a rotation angle θz described below is controlled.

The oblique-viewing endoscope 4100 is supported by a support arm device 5027. The support arm device 5027 has a function to hold the oblique-viewing endoscope 4100 instead of the scopist and to allow the oblique-viewing endoscope 4100 to be moved by an operation of the operator or the assistant so that a desired part can be observed.

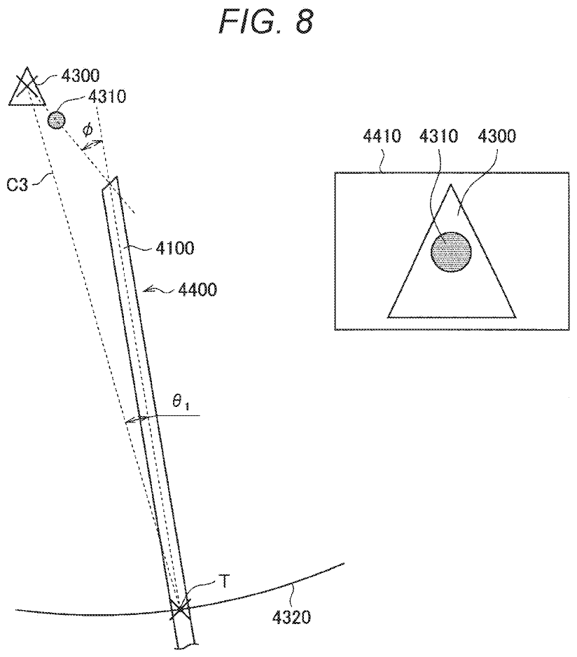

FIG. 7 is a schematic view illustrating the oblique-viewing endoscope 4100 and a forward-viewing endoscope 4150 in comparison. In the forward-viewing endoscope 4150, a direction (C1) of an objective lens toward a subject coincides with a longitudinal direction (C2) of the forward-viewing endoscope 4150. On the other hand, in the oblique-viewing endoscope 4100, the direction (C1) of the objective lens toward the subject has a predetermined angle φ with respect to the longitudinal direction (C2) of the oblique-viewing endoscope 4100.

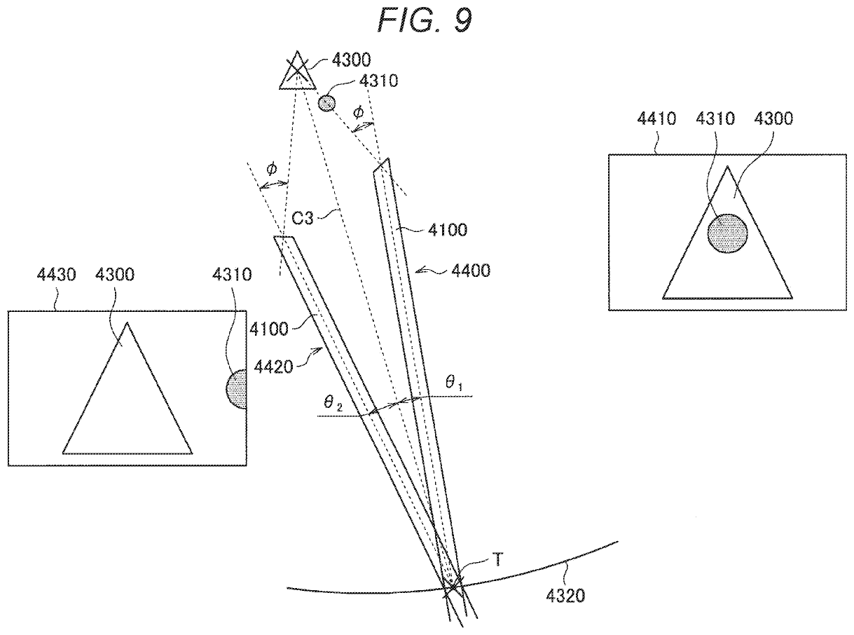

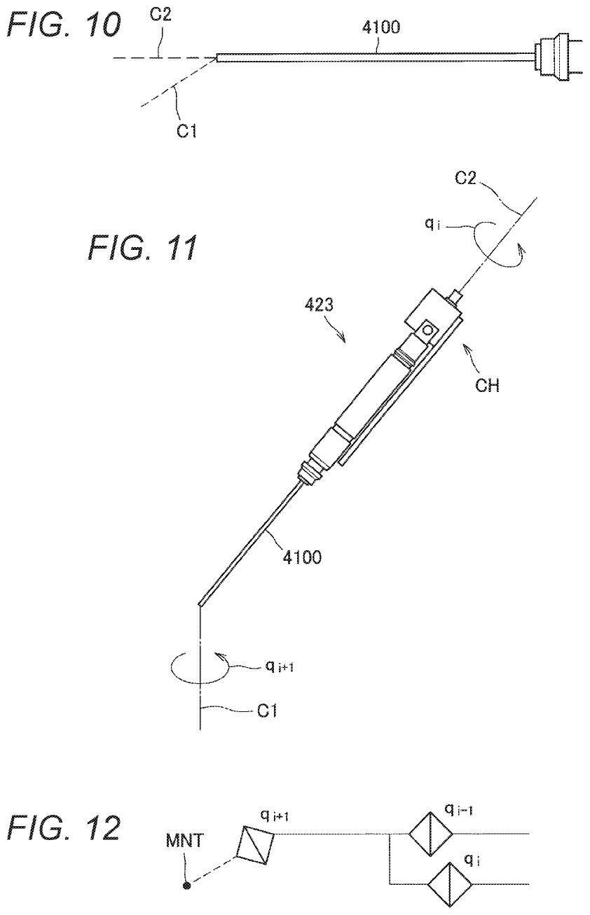

FIGS. 8 and 9 are schematic diagrams illustrating states in which the oblique-viewing endoscope 4100 is inserted through an abdominal wall 4320 into a human body, and an observation target 4300 is observed. In FIGS. 8 and 9, a trocar point T is a position where a trocar 5025a is disposed, and indicates an insertion position of the oblique-viewing endoscope 4100 into the human body. A C3 direction illustrated in FIGS. 8 and 9 is a direction connecting the trocar point T and the observation target 4300. In a case where an obstacle 4310 such as an organ is present in front of the observation target 4300, the observation target 4300 comes behind the obstacle 4310 and the entire area of the observation target 4300 cannot be observed when the observation target 4300 is observed by the forward-viewing endoscope 4150 from the C3 direction illustrated in FIGS. 8 and 9. FIG. 8 illustrates a state 4400 in which the oblique-viewing endoscope 4100 is used, and an insertion direction of the oblique-viewing endoscope 4100 is different from the C3 direction, and a captured image 4410 captured by the oblique-viewing endoscope 4100 in the case of the state 4400. Even in the case of using the oblique-viewing endoscope 4100, the observation target 4300 comes behind the obstacle 4310 in the state 4400 illustrated in FIG. 8.

Meanwhile, FIG. 9 illustrates a state 4420 in which the insertion direction of the oblique-viewing endoscope 4100 is changed from the state 4400 in FIG. 8 and the direction of the objective lens is also changed in addition to the state in FIG. 8, and a captured image 4430 in the state 4420. By changing the insertion direction of the oblique-viewing endoscope 4100 as in the state 4420 in FIG. 9, the observation target 4300 is not blocked by the obstacle 4310 and can be observed at a changed viewpoint.

### 4. CONTROL OF ARM SUPPORTING OBLIQUE-VIEWING ENDOSCOPE ACCORDING TO PRESENT EMBODIMENT

In the present embodiment, a technology that enables implementation of an oblique-viewing endoscope holder arm with maintained hand-eye coordination will be mainly described. Note that the hand-eye coordination can mean coordination of the sense of hands and the sense of eyes (vision) (matching of the sense of hands and the sense of eyes (vision)). Such a technology has “(1) modeling an oblique-viewing endoscope unit as a plurality of interlocking links” as one of characteristics. Furthermore, such a technology has “(2) extending whole body coordination control of an arm and performing control using a relationship between a relative motion space and the interlocking link” as another one of characteristics.

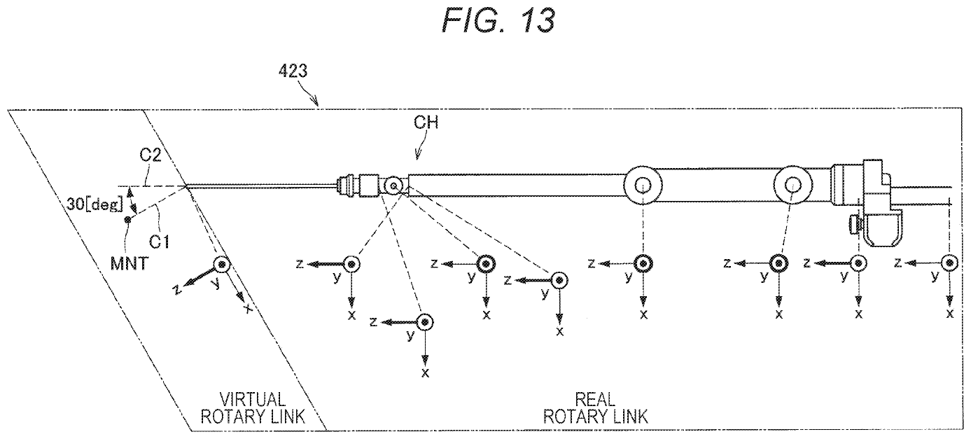

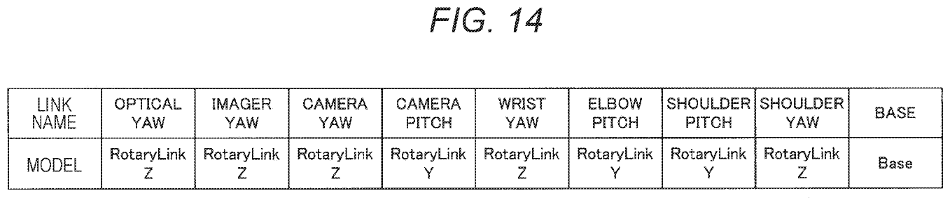

First, a use method and an operation of the oblique-viewing endoscope will be described. FIG. 10 is a view for describing an optical axis of the oblique-viewing endoscope. Referring to FIG. 10, a hard endoscope axis C2 and an oblique-viewing endoscope optical axis C1 in the oblique-viewing endoscope 4100 are illustrated. Furthermore, FIG. 11 is a view for describing an operation of the oblique-viewing endoscope. Referring to FIG. 11, the oblique-viewing endoscope optical axis C1 is inclined relative to the hard endoscope axis C2. Furthermore, referring to FIG. 11, the endoscope device 423 has a camera head CH.

Here, the scopist rotates the camera head CH to adjust a monitor screen in order to maintain the operator's hand-eye coordination with a rotation operation of the oblique-viewing endoscope during surgery. Then, when the scopist rotates the camera head CH, an arm dynamic characteristic changes around the hard endoscope axis C2. The display screen on the monitor rotates around the oblique-viewing endoscope optical axis C1. In FIG. 11, the rotation angle around the hard endoscope axis C2 is illustrated as qᵢ, and the rotation angle around the oblique-viewing endoscope optical axis C1 is illustrated as qᵢ₊₁.

Next, the above “(1) modeling an oblique-viewing endoscope unit as a plurality of interlocking links” will be described. In the present embodiment, characteristics of the operation around the hard endoscope axis C2 and the operation around the oblique-viewing endoscope optical axis C1 are modeled and control is performed. First, the oblique-viewing endoscope is modeled using a real rotary link and a virtual rotary link. Note that, in the present embodiment, description will be given mainly using the real rotary link as an example of a real link and the virtual rotary link as an example of a virtual link. However, another real link (such as a translating real link) may be used instead of the real rotary link, and another virtual link (such as a translating virtual link) may be used instead of the virtual rotary link. An axis of the real rotary link may be the hard endoscope axis C2 (=a rotation axis of an imager), and an axis of the virtual rotary link may be the oblique-viewing endoscope optical axis C1. Here, the virtual rotary link is a link that does not actually exist, and operates in conjunction with the real rotary link.

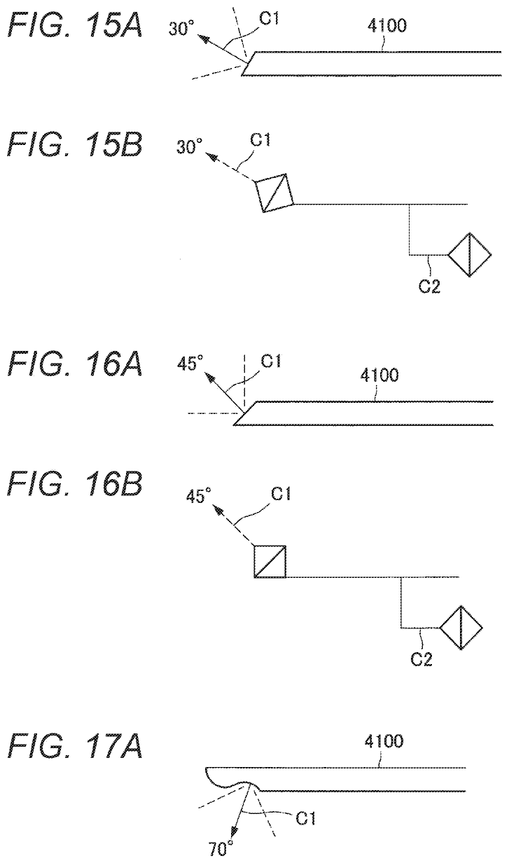

FIG. 12 is a diagram for describing modeling and control. Referring to FIG. 12, the rotation angle at each link is illustrated. Furthermore, referring to FIG. 12, a monitor coordinate system MNT is illustrated. Specifically, control is performed such that a relative motion space c represented by (13) below becomes zero.

[Math. 12]

c(=αᵢ₊₁*qᵢ₊₁+αᵢ*qᵢ)=qᵢ₊₁−qᵢ (13)

Next, the above “(2) extending whole body coordination control of an arm and performing control using a relationship between a relative motion space and the interlocking link” will be described. In the present embodiment, the whole body coordination control is performed in an integrated manner by extension using the interlocking links and the relative motion space. In a joint space, a real rotation axis and a virtual rotation axis are considered. The real rotation axis and the virtual rotation axis do not depend on an arm configuration. Furthermore, the relative motion space is taken into consideration for the motion purpose, in addition to the Cartesian space. By changing the motion purpose in the Cartesian space, various operations become possible.

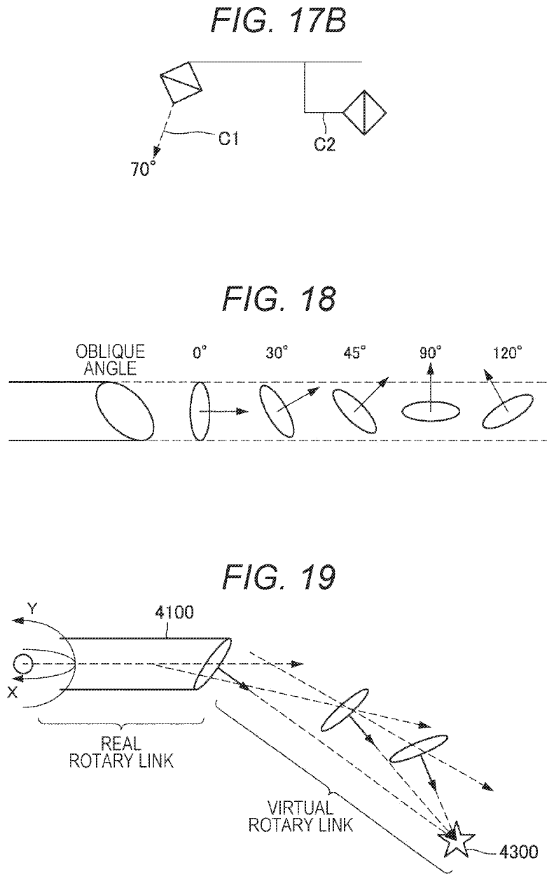

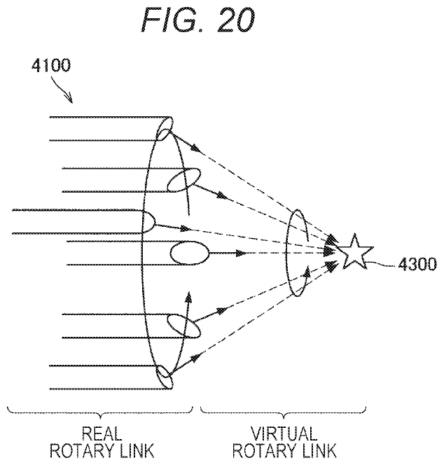

For example, assuming a case in which the extension of the whole body coordination control is applied to a six-axis arm and an oblique-viewing endoscope unit. FIG. 3 illustrates the rotation angles at respective links as q₁ to q₈. q₇ corresponds to the rotation angle around the axis of the real rotary link (=the rotation axis of the imager), and q₈ corresponds to the rotation angle around the axis of the virtual rotary link. FIGS. 13 and 14 are diagrams illustrating examples of link configurations in a case where the extension of the whole body coordination control is applied to a six-axis arm and an oblique-viewing endoscope unit. At this time, the control expression is expressed as in (14) below.

$$\begin{matrix}[ Math . 13 ] & \\ [ \begin{matrix}{\dot{q}}_{1} \\ ⋮ \\ {\dot{q}}_{7} \\ {\dot{q}}_{8}\end{matrix} ] = {J}^{\#} [ \begin{matrix}\dot{x} \\ \dot{c}\end{matrix} ] & ( 14 )\end{matrix}$$

Here, in the above (14), a time differential value of q₈ and a time differential value of the relative motion space c correspond to extended part of the whole body coordination control.

In the above, “(2) extending whole body coordination control of an arm and performing control using a relationship between a relative motion space and the interlocking link” has been described.

### 5. SETTING OF VIRTUAL LINK

Next, setting of the virtual link will be described. The arithmetic condition setting unit 242 can function as a virtual link setting unit that sets the virtual rotary link as an example of the virtual link. For example, the arithmetic condition setting unit 242 sets the virtual link by setting at least one of a distance or a direction of the virtual link. FIG. 13 illustrates example of the “virtual rotary link” and the “real rotary link”. As illustrated in FIG. 13, the real rotary link is a link corresponding to a lens barrel axis of a scope. The virtual rotary link is a link corresponding to the oblique-viewing endoscope optical axis C1 of the scope.

The arithmetic condition setting unit 242 models the virtual rotary link on the basis of a coordinate system defined on the basis of a distal end of the real rotary link of the arm, an arbitrary point existing on the oblique-viewing endoscope optical axis C1, and a line connecting the aforementioned points, and uses the whole body coordination control. Thereby, realization of the motion purposes such as posture fixation in the virtual rotary link coordinate system, and fixation of the viewpoint in a direction of an arbitrary point existing at the distal end of the virtual rotary link while maintaining the position of the trocar point to serve as the scope insertion position during surgery becomes possible without depending on the hardware configuration of the arm. Note that the distal end of the real rotary link can mean a point where the optical axis C1 on the arm passes.

The arithmetic condition setting unit 242 can set the virtual rotary link on the basis of a scope specification to be connected or an arbitrary point in the space. According to the setting of the virtual rotary link based on the scope specification, it is not necessary to limit the conditions in which the virtual rotary link is set for a case of using a specific scope. Therefore, an operation of the motion purpose can be realized only by dynamic model updating by the virtual rotary link setting at the time of changing the scope.

The scope specification may include at least one of a structural specification of the scope or a functional specification of the scope. At this time, the structural specification of the scope may include at least one of an oblique angle of the scope or a dimension of the scope. The scope specification may include the position of the scope's axis (information regarding the scope's axis can be used to set the real rotary link). Furthermore, the functional specification of the scope may include a focus distance of the scope.

For example, in the case of the virtual rotary link setting based on the scope specification, the direction of the virtual rotary link that will be a connection link from the distal end of the real rotary link can be determined from oblique angle information. Furthermore, the distance to the virtual rotary link to be connected to the distal end of the real rotary link can be determined from scope dimension information. The length of the virtual rotary link can be determined to set a focus point as a fixation target of the motion purpose from focus distance information. As a result, the operation of the motion purpose corresponding to change in various types of scopes can be realized only by changing the setting of the virtual rotary link, using the same control algorithm.

Furthermore, in the case of changing the scope, the above virtual rotary link can be dynamically changed as a virtual link not depending on the hardware configuration of the arm. For example, in the case of changing the scope from an oblique-viewing endoscope with 30-degree oblique angle to an oblique-viewing endoscope with 45-degree oblique angle, a new virtual rotary link can be reset on the basis of the scope specification after change. Thereby, switching of the motion purpose according to the scope change becomes possible.

The virtual rotary link setting based on the scope specification is updated when the scope specification information is set to the arm system. However, information input means to the arm system is not limited. For example, the arithmetic condition setting unit 242 can recognize a scope ID corresponding to the scope at the time of connection of the scope, and acquire the specification of the scope corresponding to the recognized scope ID.

At this time, in a case where the scope ID is written in a memory of the scope, the arithmetic condition setting unit 242 may recognize the scope ID read from the memory. In such a case, the virtual rotary link is updated even if the scope specification after change is not input from the user. Thus, surgery can be smoothly continued. Alternatively, in a case where the scope ID is written on a surface of the scope, for example, the user who saw the scope ID inputs the scope ID as input information via the input unit 210, and the arithmetic condition setting unit 242 may recognize the scope ID on the basis of the input information.

Furthermore, the scope specification corresponding to the scope ID may be obtained from anywhere. For example, in a case where the scope specification is stored in a memory in the arm system, the scope specification may be obtained from the memory in the arm system. Alternatively, in a case where the scope specification is stored in an external device connected to a network, the scope specification may be acquired via the network. The virtual rotary link can be automatically set on the basis of the scope specification acquired in this manner.

In the virtual rotary link, it is also conceivable to set an arbitrary point of the observation target present at an arbitrary distance from the distal end of the connected scope as a distal end of the virtual rotary link. Therefore, the arithmetic condition setting unit 242 may set or change the virtual rotary link on the basis of the distance or the direction from the distal end of the scope to the observation target obtained from the sensor. In the case where the position of the observation target dynamically changes, the arithmetic condition setting unit 242 may acquire direction and distance information with respect to the distal end of the scope on the basis of sensor information for specifying a spatial position of the observation target, and set or update the virtual rotary link on the basis of the information. An operation of attention can be realized while switching the observation target during surgery in response to an operation request of continuously giving close attention to the observation target.

The type of sensor is not particularly limited. For example, the sensor may include at least one of a distance measurement sensor, a visible light sensor, or an infrared sensor. Furthermore, the sensor information may be acquired in any way.

For example, in a case of using a user interface (UI), position information of an arbitrary point on a monitor or three-dimensional data may be able to be determined by allowing the user to directly specify the arbitrary point. By the direct operation of the user, any portion or point can be intuitively specified as the observation target. In other words, in a case where coordinates on an image displayed on the display device 30 are input via the input unit 210, the arithmetic condition setting unit 242 may determine the observation target on the basis of the coordinates and set the virtual rotary link on the basis of the distance or the direction from the observation target to the distal end of the scope. In this case, the direct specification may be performed by any operation, by a touch operation on the screen, by a gaze operation with a line of sight, or the like.

Furthermore, in the case of using an image recognition technology, the position of a specific observation target can be automatically recognized from 2D or 3D video information, and a spatial position can be specified. In other words, the arithmetic condition setting unit 242 may set the virtual rotary link on the basis of the distance or the direction (from the observation target to the distal end of the scope) recognized by the image recognition.

In the case of using the observation target spatial position specification technology by the image recognition, the position may be acquired in real time even in a case where the observation target dynamically moves. In other words, the arithmetic condition setting unit 242 may dynamically update the virtual rotary link on the basis of the distance or the direction (from the observation target to the distal end of the scope) dynamically recognized by the image recognition. Thereby, the distal end of the virtual rotary link can be updated in real time. For example, in the case of the observation target with motion, the continuous attention to the observation target becomes possible by continuously recognizing the observation target by the image recognition.

For example, the arithmetic condition setting unit 242 calculates an arm posture change amount for continuing the motion purpose such as posture fixation or viewpoint fixation based on the information of the distal end of the virtual rotary link by the whole body coordination control, and may reflect the calculated result as a rotation command of each real rotary link on the arm. Follow-up of the observation target (in particular, follow-up of the forceps during surgery, or the like) can be realized. In other words, the motion purpose of keeping capturing the observation target at the center of the virtual rotary link can be realized by the control of the real rotary link.

Furthermore, in the case of surgery, a spatial position of a specific part of a patient can be specified using a navigation system or a CT device. In other words, the arithmetic condition setting unit 242 may set the virtual rotary link on the basis of the distance or the direction (from the observation target to the distal end of the scope) recognized by the navigation system or the CT device. Thereby, an arbitrary motion purpose based on the relationship between the specific part and the scope can be realized in accordance with a surgical purpose.

Moreover, the spatial position of the specific part of the patient can be specified in real time during surgery by combining the patient coordinate information acquired by the CT device, an MRI device, or the like before surgery with the navigation system or the CT device during surgery. In other words, the arithmetic condition setting unit 242 may dynamically update the virtual rotary link on the basis of the patient coordinate information acquired by the CT device or the MRI device before surgery, and the distance and the direction (from the observation target to the distal end of the scope) dynamically recognized by the navigation system or the CT device during surgery. Thereby, an arbitrary motion purpose based on the relationship between the specific part and the scope can be realized in accordance with a surgical purpose.

Furthermore, the spatial position of the distal end of the real rotary link of the arm changes with the movement or change in posture of the arm. However, in a case where the observation target located at the distal end of the virtual rotary link of the arm is stationary, the motion purpose of maintaining the observation target at the distal end of the virtual rotary link by updating the length of the virtual rotary link (the distance between the distal end of the real rotary link of the arm and the observation target) may be realized. In other words, the arithmetic condition setting unit 242 may dynamically update the virtual rotary link according to a moving amount or the posture of the arm. Thereby, the user can continuously observe the observation target.

In the above description, a case where the scope is the oblique-viewing endoscope has been mainly assumed. However, as described above, the oblique angle of the scope can be arbitrarily changed on the basis of the scope specification. Therefore, the scope may be a forward-viewing endoscope or a side-viewing endoscope. In other words, the arithmetic condition setting unit 242 can change the setting of the virtual rotary link corresponding to switching of the endoscope having an arbitrary oblique angle (including the forward-viewing endoscope, the oblique-viewing endoscope, and the side-viewing endoscope). Alternatively, (an oblique angle variable oblique-viewing endoscope) capable of changing the oblique angle within the same device exists as the endoscope having an arbitrary oblique angle. Therefore, as the scope, the oblique angle variable oblique-viewing endoscope may be used. Although the oblique angle is usually changed by scope switching, the oblique angle can be changed within the same device, using the oblique angle variable oblique-viewing endoscope.

FIG. 18 is a diagram for describing an oblique angle variable oblique-viewing endoscope. Referring to FIG. 18, a state in which the oblique angle of the oblique angle variable oblique-viewing endoscope can be changed among 0°, 30°, 45°, 90°, and 120°. However, the change range of the oblique angle of the oblique angle variable oblique-viewing endoscope is not limited to these angles. An arbitrary motion purpose due to setting change in the virtual rotary link can be realized by detecting the oblique angle information after change by the system or inputting the oblique angle information after change to the arm system, similarly to at the time of switching the oblique-viewing endoscope.

Generally, in use cases such as a zoom operation to change an insertion amount into a body of the oblique-viewing endoscope, and a scope rotation operation to change a field of view direction of the oblique-viewing endoscope, it is difficult to keep the observation target in the center of the camera in a case where the operation is performed on the basis of only the real rotary link information of the arm without considering the optical axis direction of the oblique-viewing endoscope.

In contrast, by modeling the virtual rotary link having the observation target at the distal end, the operation of attention to the distal end of the virtual rotary link may be provided as the motion purpose while maintaining the connection relationship between the real rotary link of the arm and the virtual rotary link to be connected thereto (corresponding to the oblique angle in the case of the oblique-viewing endoscope). In other words, the arithmetic condition setting unit 242 may dynamically update the virtual rotary link on the basis of the zoom operation or the rotation operation of the oblique-viewing endoscope. Such an example will be described with reference to FIGS. 19 and 20.

FIG. 19 is a diagram for describing update of a virtual rotary link in consideration of a zoom operation of the oblique angle fixed oblique-viewing endoscope. Referring to FIG. 19, an oblique angle fixed oblique-viewing endoscope 4100 and the observation target 4300 are illustrated. For example, as illustrated in FIG. 19, in a case where the zoom operation is performed, the arithmetic condition setting unit 242 changes the distance and the direction of the virtual rotary link (makes the distance of the virtual rotary link short and largely inclines the direction of the virtual rotary link with respect to the scope axis in a case of an enlargement operation as illustrated in FIG. 19), whereby the observation target 4300 is captured in the center of the camera, and the motion purpose can be realized. Note that, in the case of the oblique angle variable oblique-viewing endoscope, the observation target 4300 can be captured in the center of the camera at the zoom operation. In other words, the arithmetic condition setting unit 242 changes the oblique angle and the distance of the virtual rotary link in the state where the direction (posture) of the virtual rotary link is fixed in the case of the zoom operation, whereby the observation target 4300 is captured in the center of the camera, and the motion purpose can be realized.

FIG. 20 is a diagram for describing update of a virtual rotary link in consideration of a zoom operation of the oblique angle fixed oblique-viewing endoscope. Referring to FIG. 20, an oblique angle fixed oblique-viewing endoscope 4100 and the observation target 4300 are illustrated. For example, as illustrated in FIG. 20, the arithmetic condition setting unit 242 changes the direction (posture) of the virtual rotary link in a state where the distance between the oblique angle and the virtual rotary link is fixed, as illustrated in FIG. 20, in the case of the rotation operation, whereby the observation target 4300 is captured in the center of the camera, and the motion purpose can be realized. Note that, in the case of the oblique angle variable oblique-viewing endoscope, the observation target 4300 can be captured in the center of the camera at the rotation operation. In other words, the arithmetic condition setting unit 242 changes the oblique angle in the state where the distance of the virtual rotary link and the direction (posture) of the virtual rotary link are fixed in the case of the rotation operation, whereby the observation target 4300 is captured in the center of the camera, and the motion purpose can be realized.

In the examples illustrated in FIGS. 19 and 20, a case where the observation target is stationary has been mainly assumed. However, a case where the observation target moves is also assumed. In such a case, the follow-up of the observation target, as described above, and the motion purpose of the zoom operation or the rotation operation based on the following observation target can be realized in combination. In other words, the arithmetic condition setting unit 242 may dynamically update the virtual rotary link on the basis of the distance or the direction (from the observation target to the distal end of the scope) dynamically recognized by the image recognition, and the zoom operation or the rotation operation of the scope.

The setting of the virtual rotary link has been described above.

### 6. CONCLUSION

According to the present embodiment, provided is a medical support arm system including an articulated arm (arm unit 120) configured to support a scope that acquires an image of an observation target in an operation field, and a control unit (arm control unit 110) configured to control the articulated arm on the basis of a relationship between a real link corresponding to a lens barrel axis of the scope and a virtual link corresponding to an optical axis of the scope. According to such a configuration, the arm unit 120 can be controlled to maintain the hand-eye coordination in the case of using the arm unit 120 supporting the oblique-viewing endoscope.

More specifically, according to the present embodiment, the oblique-viewing endoscope is modeled as a plurality of interlocking links of the axis of the real rotary link and the axis of the virtual rotary link, and the whole body coordination control is used in consideration of the model, whereby control not depending on the motion purpose and the arm configuration becomes possible. In particular, by giving a posture fixing command in the monitor coordinate system to the motion purpose, an arm operation with maintained hand-eye coordination can be realized.

The type of the endoscope applicable to the present embodiment is not particularly limited. It is sufficient that the oblique-viewing endoscope model is set in the arm system when attaching the endoscope.

FIGS. 15A and 15B are diagrams illustrating a first example of an oblique-viewing endoscope applicable to the present embodiment. As illustrated in FIGS. 15A and 15B, the oblique-viewing endoscope according to the present embodiment may be an oblique-viewing endoscope with an oblique angle of 30°.

FIGS. 16A and 16B are diagrams illustrating a second example of an oblique-viewing endoscope applicable to the present embodiment. As illustrated in FIGS. 16A and 16B, the oblique-viewing endoscope according to the present embodiment may be an oblique-viewing endoscope with an oblique angle of 45°.

FIGS. 17A and 17B are diagrams illustrating a third example of an oblique-viewing endoscope applicable to the present embodiment. As illustrated in FIGS. 17A and 17B, the oblique-viewing endoscope according to the present embodiment may be a side-viewing endoscope with an oblique angle of 70°.

Although the favorable embodiments of the present disclosure have been described in detail with reference to the accompanying drawings, the technical scope of the present disclosure is not limited to such examples. It is obvious that persons having ordinary knowledge in the technical field of the present disclosure can conceive various modifications or alterations within the scope of the technical idea described in the claims, and the modifications and alterations are naturally understood to belong to the technical scope of the present disclosure.

Furthermore, the effects described in the present specification are merely illustrative or exemplary and are not restrictive. That is, the technology according to the present disclosure can exhibit other effects obvious to those skilled in the art from the description of the present specification together with or in place of the above-described effects.

Note that following configurations also belong to the technical scope of the present disclosure.
(1) A medical support arm system including:

an articulated arm configured to support a scope that acquires an image of an observation target in an operation field; and

a control unit configured to control the articulated arm on the basis of a relationship between a real link corresponding to a lens barrel axis of the scope and a virtual link corresponding to an optical axis of the scope.

(2)

The medical support arm system according to (1), further including:

a virtual link setting unit configured to set the virtual link.

(3)

The medical support arm system according to (2), in which

the virtual link setting unit sets the virtual link on the basis of a specification of the scope.

(4)

The medical support arm system according to (3), in which

the specification of the scope includes at least one of a structural specification of the scope or a functional specification of the scope.

(5)

The medical support arm system according to (4), in which

the structural specification includes at least one of an oblique angle of the scope or a dimension of the scope, and the functional specification includes a focus distance of the scope.

(6)

The medical support arm system according to (4) or (5), in which

the virtual link setting unit recognizes a scope ID corresponding to the scope and acquires the specification of the scope corresponding to the recognized scope ID.

(7)

The medical support arm system according to (6), in which

the virtual link setting unit recognizes the scope ID written in a memory of the scope.

(8)

The medical support arm system according to (6), in which

the virtual link setting unit recognizes the scope ID on the basis of input information from a user.

(9)

The medical support arm system according to any one of (2) to (8), in which

the virtual link setting unit sets the virtual link on the basis of a distance or a direction from a distal end of the scope to the observation target obtained from a sensor.

(10)

The medical support arm system according to (9), in which,

in a case where coordinates of the image to be displayed by a display device are input via an input device, the virtual link setting unit determines the observation target on the basis of the coordinates, and sets the virtual link on the basis of the distance or the direction from the observation target to the distal end of the scope.

(11)

The medical support arm system according to (10), including:

at least one of the display device or the input device.

(12)

The medical support arm system according to (9), in which

the virtual link setting unit sets the virtual link on the basis of the distance or the direction recognized by image recognition.

(13)

The medical support arm system according to (12), in which

the virtual link setting unit dynamically updates the virtual link on the basis of the distance or the direction dynamically recognized by the image recognition.

(14)

The medical support arm system according to (9), in which

the virtual link setting unit sets the virtual link on the basis of the distance or the direction recognized by a navigation system or a CT device.

(15)

The medical support arm system according to (14), in which

the virtual link setting unit dynamically updates the virtual link on the basis of patient coordinate information acquired by a CT device or an MRI device before surgery, and the distance or the direction dynamically recognized by the navigation system or the CT device during surgery.

(16)

The medical support arm system according to any one of (2) to (15), in which

the virtual link setting unit dynamically updates the virtual link according to a moving amount or a posture of the articulated arm.

(17)

The medical support arm system according to any one of (2) to (16), in which

the virtual link setting unit sets the virtual link by setting at least one of a distance or a direction of the virtual link.

(18)

The medical support arm system according to any one of (1) to (17), in which

the scope is a forward-viewing endoscope, an oblique-viewing endoscope, or a side-viewing endoscope.

(19)

The medical support arm system according to any one of (1) to (17), in which

the scope is an oblique angle variable endoscope.

(20)

The medical support arm system according to any one of (2) to (16), in which

the virtual link setting unit dynamically updates the virtual link on the basis of a zoom operation or a rotation operation of the scope.

(21)

The medical support arm system according to (12), in which

the virtual link setting unit dynamically updates the virtual link on the basis of the distance or the direction dynamically recognized by the image recognition, and the zoom operation or the rotation operation of the scope.

(22)

A control device including:

a control unit configured to control an articulated arm that supports a scope on the basis of a relationship between a real link corresponding to a lens barrel axis of the scope and a virtual link corresponding to an optical axis of the scope.

### REFERENCE SIGNS LIST

1 Robot arm control system
10 Robot arm device
20 Control device
30 Display device
110 Arm control unit
111 Drive control unit
120 Arm unit
130 Joint unit
131 Joint drive unit
132 Indirect state detection unit
133 Rotation angle detection unit
134 Torque detection unit
140 Imaging unit
210 Input unit
220 Storage unit
230 Control unit
240 Whole body coordination control unit
241 Arm state acquisition unit
242 Arithmetic condition setting unit
243 Virtual force calculation unit
244 Real force calculation unit
250 Ideal joint control unit
251 Disturbance estimation unit
252 Command value calculation unit

1. A medical support arm system comprising:
an articulated arm configured to support a scope that acquires an image of an observation target in an operation field; and
a control unit configured to control the articulated arm on a basis of a relationship between a real link corresponding to a lens barrel axis of the scope and a virtual link corresponding to an optical axis of the scope.

2. The medical support arm system according to claim 1, further comprising:
a virtual link setting unit configured to set the virtual link.

3. The medical support arm system according to claim 2, wherein
the virtual link setting unit sets the virtual link on a basis of a specification of the scope.

4. The medical support arm system according to claim 3, wherein
the specification of the scope includes at least one of a structural specification of the scope or a functional specification of the scope.

5. The medical support arm system according to claim 4, wherein
the structural specification includes at least one of an oblique angle of the scope or a dimension of the scope, and the functional specification includes a focus distance of the scope.

6. The medical support arm system according to claim 3, wherein
the virtual link setting unit recognizes a scope ID corresponding to the scope and acquires the specification of the scope corresponding to the recognized scope ID.

7. The medical support arm system according to claim 6, wherein
the virtual link setting unit recognizes the scope ID written in a memory of the scope.

8. The medical support arm system according to claim 6, wherein
the virtual link setting unit recognizes the scope ID on a basis of input information from a user.

9. The medical support arm system according to claim 2, wherein
the virtual link setting unit sets the virtual link on a basis of a distance or a direction from a distal end of the scope to the observation target obtained from a sensor.

10. The medical support arm system according to claim 9, wherein,
in a case where coordinates of the image to be displayed by a display device are input via an input device, the virtual link setting unit determines the observation target on a basis of the coordinates, and sets the virtual link on a basis of the distance or the direction from the observation target to the distal end of the scope.

11. The medical support arm system according to claim 10, comprising:
at least one of the display device or the input device.

12. The medical support arm system according to claim 9, wherein
the virtual link setting unit sets the virtual link on a basis of the distance or the direction recognized by image recognition.

13. The medical support arm system according to claim 12, wherein
the virtual link setting unit dynamically updates the virtual link on a basis of the distance or the direction dynamically recognized by the image recognition.

14. The medical support arm system according to claim 9, wherein
the virtual link setting unit sets the virtual link on a basis of the distance or the direction recognized by a navigation system or a CT device.

15. The medical support arm system according to claim 14, wherein
the virtual link setting unit dynamically updates the virtual link on a basis of patient coordinate information acquired by a CT device or an MRI device before surgery, and the distance or the direction dynamically recognized by the navigation system or the CT device during surgery.

16. The medical support arm system according to claim 2, wherein
the virtual link setting unit dynamically updates the virtual link according to a moving amount or a posture of the articulated arm.

17. The medical support arm system according to claim 2, wherein
the virtual link setting unit sets the virtual link by setting at least one of a distance or a direction of the virtual link.

18. The medical support arm system according to claim 1, wherein
the scope is a forward-viewing endoscope, an oblique-viewing endoscope, or a side-viewing endoscope.

19. The medical support arm system according to claim 1, wherein
the scope is an oblique angle variable endoscope.

20. The medical support arm system according to claim 2, wherein
the virtual link setting unit dynamically updates the virtual link on a basis of a zoom operation or a rotation operation of the scope.

21. The medical support arm system according to claim 12, wherein
the virtual link setting unit dynamically updates the virtual link on a basis of the distance or the direction dynamically recognized by the image recognition, and the zoom operation or the rotation operation of the scope.

22. A control device comprising:
a control unit configured to control an articulated arm that supports a scope on a basis of a relationship between a real link corresponding to a lens barrel axis of the scope and a virtual link corresponding to an optical axis of the scope.
